(19) United States
(12) Patent Application Publication
(10) Pub. No.: US 2022/0089550 A1
(43) Pub. Date: Mar. 24, 2022

(54) CRYSTALLINE FORMS OF (S)-2-(7-CYANO-1H-BENZIMIDAZOL-1 YL)-N-{1-[4-(1-CYANO-1-METHYLETHYL)PHENYL]ETHYL}ACETAMIDE

(71) Applicant: NEOMED INSTITUTE - INSTITUT NEOMED, Montreal, CA (US)

(72) Inventors: Jilong JIANG, Markham (CA); Alan TSE, Markham (CA)

(21) Appl. No.: 17/421,674

(22) PCT Filed: Jan. 7, 2020
(86) PCT No.: PCT/CA2020/050016

§ 371 Date: Jul. 8, 2021

Related U.S. Application Data
(60) Provisional application No. 62/789,740, filed on Jan. 8, 2019.

(51) Int. Cl. C07D 235/16

(52) U.S. Cl. CPC C07D 235/16; C07B 2200/13

(57) ABSTRACT

Crystalline Forms of a compound of Formula I are provided.

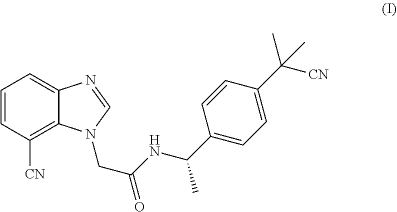 [SMILES: C[C@H](NC(=O)Cn1cnc2cccc(C#N)c21)c1ccc(C(C)(C)C#N)cc1]

Crystalline Form A is among the crystalline Forms identified. Form A has an X-ray powder diffraction (XRPD) pattern having characteristic peaks expressed in degrees 2Θ (±0.2° 2Θ) at 3.07, 5.96, 11.89 and 17.85, and a Differential Scanning Calorimetry (DSC) thermogram that exhibits an endotherm having a peak temperature of about 168.9° C.

### RELATED APPLICATION

This application is a US National Stage of PCT Application No. PCT/CA2020/050016, filed on Jan. 7, 2020, which claims priority to U.S. provisional application No. 62/789,740 filed on Jan. 8, 2019. The entire content of each of the aforementioned patent applications is incorporated herein by reference for all purposes.

### TECHNICAL FIELD

The technical field relates to crystalline forms of the compound (S)-2-(7-Cyano-1H-benzimidazol-1-yl)-N-{1-[4-(1-cyano-1-methylethyl)phenyl]ethyl}acetamide, as well as pharmaceutical compositions, therapeutic uses thereof and processes of manufacture.

### BACKGROUND

Pain sensation in mammals is due to the activation of the peripheral terminals of a specialized population of sensory neurons known as nociceptors. Capsaicin, the active ingredient in hot peppers, produces sustained activation of nociceptors and also produces a dose-dependent pain sensation in humans. Cloning of the vanilloid receptor 1 (VR1 or TRPV1) demonstrated that VR1 is the molecular target for capsaicin and its analogues. (Caterina, M. J., Schumacher, M. A., et. al. Nature (1997) v.389 p 816-824). Functional studies using VR1 indicate that it is also activated by noxious heat, tissue acidification and other inflammatory mediators (Tominaga, M., Caterina, M. J. et. al. Neuron (1998) v.21, p. 531-543). Expression of VR1 is also regulated after peripheral nerve damage of the type that leads to neuropathic pain. These properties of VR1 make it a highly relevant target for pain and for diseases involving inflammation. While agonists of the VR1 receptor can act as analgesics through nociceptor destruction, the use of agonists, such as capsaicin and its analogues, is limited due to their pungency, neurotoxicity and induction of hypothermia. Instead, agents that block the activity of VR1 should prove more useful. Antagonists would maintain the analgesic properties but avoid pungency and neurotoxicity side effects.

(S)-2-(7-Cyano-1H-benzimidazol-1-yl)-N-{1-[4-(1-cyano-1-methylethyl) phenyl]ethyl}acetamide (also referred to herein as Compound 1) was found to inhibit the activity at the VR1 receptor (WO 2008018827). Compound 1 was found to be an antagonist of capsaicin responses with an IC50 of 41-49 nM using the 384 plate-based imaging assay that monitors drug induced intracellular Ca²⁺ level in whole cells described on pages 117-118 of WO 2008018827.

It is desirable to identify stable crystalline forms of this compound, that may be suitable for therapeutic use.

### SUMMARY

In one aspect, there is provided a compound of Formula I:

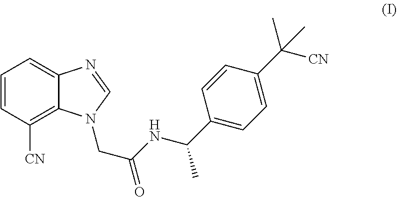 [SMILES: C[C@H](NC(=O)Cn1cnc2cccc(C#N)c21)c1ccc(C(C)(C)C#N)cc1]

which is crystalline and exhibits an X-ray powder diffraction (XRPD) pattern having characteristic peaks expressed in degrees 2Θ (±0.2° 2Θ) at 3.07, 5.96, 11.89, and 17.85.

In some embodiments, the XRPD pattern further has characteristic peaks expressed in degrees 2Θ (±0.2° 2Θ) at 23.86 and 24.63.

In some embodiments, the XRPD pattern further has characteristic peaks expressed in degrees 2Θ (±0.2° 2Θ) at 13.35, 14.90, 16.67, 20.08, 20.83, and 26.88.

In some embodiments, the XRPD pattern further has characteristic peaks expressed in degrees 2Θ (±0.2° 2Θ) at 8.92, 13.75, 22.15 and 39.20.

In one aspect, there is provided a compound of Formula I:

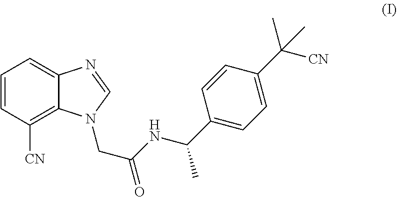 [SMILES: C[C@H](NC(=O)Cn1cnc2cccc(C#N)c21)c1ccc(C(C)(C)C#N)cc1]

which is crystalline and has a Differential Scanning Calorimetry (DSC) thermogram that exhibits an endotherm having a peak temperature of about 168.9° C.

In one aspect, there is provided a compound of Formula I:

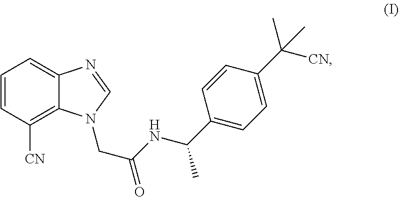 [SMILES: C[C@H](NC(=O)Cn1cnc2cccc(C#N)c21)c1ccc(C(C)(C)C#N)cc1]

having an X-ray powder diffraction pattern substantially the same as shown in FIG. 1.

In one aspect, there is provided a compound of Formula I:

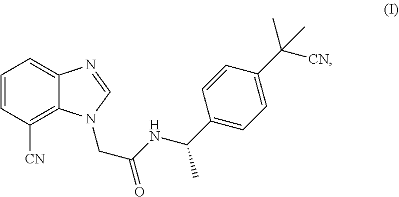 [SMILES: C[C@H](NC(=O)Cn1cnc2cccc(C#N)c21)c1ccc(C(C)(C)C#N)cc1]

which is crystalline.

In some embodiments, the compound provided herein includes one crystalline form at a purity of 95% or higher, 99% or higher, or 99.8% or higher.

In some embodiments, the compound provided herein is substantially pure.

In one aspect, there is provided a compound of Formula I:

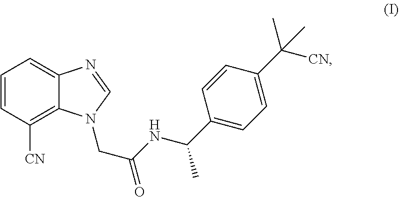 [SMILES: C[C@H](NC(=O)Cn1cnc2cccc(C#N)c21)c1ccc(C(C)(C)C#N)cc1]

which is a solid comprising:
a first crystalline form that exhibits an X-ray powder diffraction (XRPD) pattern having characteristic peaks expressed in degrees 2Θ (±0.2° 2Θ) at 3.07, 5.96, 11.89, 17.85, 23.86 and 24.63; and
a second crystalline form.

In some embodiments, the second crystalline form exhibits an XRPD pattern having characteristic peaks expressed in degrees 2Θ (±0.2° 2Θ) at 4.77, 12.61, 14.05, 14.41, 16.68 and 17.06.

In some embodiments, the second crystalline form exhibits an XRPD pattern having characteristic peaks expressed in degrees 2Θ (±0.2° 2Θ) at 3.86, 4.52, 6.97, 12.44, 13.50 and 13.81.

In some embodiments, the second crystalline form exhibits an XRPD pattern having characteristic peaks expressed in degrees 2Θ (±0.2° 2Θ) at 4.38, 7.78, 8.73, 10.47, 12.26, 21.08 and 23.21.

In some embodiments, the second crystalline form exhibits an XRPD pattern having characteristic peaks expressed in degrees 2Θ (±0.2° 2Θ) at 4.24, 4.92, 8.15, 8.44, 8.73, 11.98 and 15.31.

In some embodiments, the second crystalline form exhibits an XRPD pattern having characteristic peaks expressed in degrees 2Θ (±0.2° 2Θ) at 11.67, 13.09, 13.48, 14.06, 14.70 and 15.56.

In some embodiments, the first crystalline form makes up for at least 80 wt % of the solid.

In some embodiments, the first crystalline form makes up for at least 95 wt % of the solid.

In some embodiments, the first crystalline form makes up for at least 99 wt % of the solid.

In some embodiments, there is provided the use of a compound described herein, for the treatment of a nociceptive pain disorder.

In some embodiments, there is provided the use of a compound described herein, for the treatment of a chronic nociceptive pain disorder.

In some embodiments, there is provided the use of a compound described herein, for the treatment of osteoarthritis.

In some embodiments, there is provided the use of a compound described herein, for the treatment of tendinitis.

In some embodiments, there is provided the use of a compound described herein, for the treatment of chronic tendinitis.

In some embodiments, there is provided the use of a compound described herein, for the treatment of pelvic pain.

In some embodiments, there is provided the use of a compound described herein, for the treatment of neuropathic pain.

In some embodiments, there is provided the use of a compound described herein, for the treatment of peripheral neuropathy.

In some embodiments, there is provided the use of a compound described herein, for the treatment of postherpetic neuralgia (PHN).

In some embodiments, there is provided the use of a compound described herein, for the treatment of gastroesophageal reflux disease (GERD).

In some embodiments, there is provided the use of a compound described herein, for the treatment of diabetes.

In some embodiments, there is provided the use of a compound described herein, for the treatment of obesity.

In some embodiments, there is provided the use of a compound described herein, for the treatment of chronic cough.

In some embodiments, there is provided the use of a compound described herein, for the treatment of chronic obstructive pulmonary disease (COPD).

In some embodiments, there is provided the use of a compound described herein, for the treatment of irritable bowel syndrome (IBS).

In some embodiments, there is provided the use of a compound described herein, for the treatment of overactive bladder.

In some embodiments, there is provided the use of a compound described herein, for inhibiting vanilloid receptor 1 (VR1).

In some embodiments, there is provided a method for the treatment of a nociceptive pain disorder, comprising administering a compound described herein to a subject in need thereof.

In some embodiments, there is provided a method for the treatment of a chronic nociceptive pain disorder, comprising administering a compound described herein to a subject in need thereof.

In some embodiments, there is provided a method for the treatment of osteoarthritis, comprising administering a compound described herein to a subject in need thereof.

In some embodiments, there is provided a method for the treatment of tendinitis, comprising administering a compound described herein to a subject in need thereof.

In some embodiments, there is provided a method for the treatment of chronic tendinitis, comprising administering a compound described herein to a subject in need thereof.

In some embodiments, there is provided a method for the treatment of pelvic pain, comprising administering a compound described herein to a subject in need thereof.

In some embodiments, there is provided a method for the treatment of neuropathic pain, comprising administering a compound described herein to a subject in need thereof.

In some embodiments, there is provided a method for the treatment of peripheral neuropathy, comprising administering a compound described herein to a subject in need thereof.

In some embodiments, there is provided a method for the treatment of postherpetic neuralgia (PHN), comprising administering a compound described herein to a subject in need thereof.

In some embodiments, there is provided a method for the treatment of gastroesophageal reflux disease (GERD), comprising administering a compound described herein to a subject in need thereof.

In some embodiments, there is provided a method for the treatment of diabetes, comprising administering a compound described herein to a subject in need thereof.

In some embodiments, there is provided a method for the treatment of obesity, comprising administering a compound described herein to a subject in need thereof.

In some embodiments, there is provided a method for the treatment of chronic cough, comprising administering a compound described herein to a subject in need thereof.

In some embodiments, there is provided a method for the treatment of chronic obstructive pulmonary disease (COPD), comprising administering a compound described herein to a subject in need thereof.

In some embodiments, there is provided a method for the treatment of irritable bowel syndrome (IBS), comprising administering a compound described herein to a subject in need thereof.

In some embodiments, there is provided a method for the treatment of overactive bladder, comprising administering a compound described herein to a subject in need thereof.

In some embodiments, there is provided a method for inhibiting vanilloid receptor 1 (VR1), comprising administering a compound described herein to a subject in need thereof.

In some embodiments, there is provided a pharmaceutical composition, comprising a compound described herein and a pharmaceutically acceptable carrier or excipient.

In some embodiments, the pharmaceutical composition is formulated as an oral dosage form.

In some embodiments, the oral dosage form is a tablet, a capsule, a lozenge, a pastille or a granule.

In some embodiments, the pharmaceutical composition is formulated as an oral suspension.

In some embodiments, there is provided the use of a pharmaceutical composition described herein, for the treatment of a nociceptive pain disorder.

In some embodiments, there is provided the use of a pharmaceutical composition described herein, for the treatment of a chronic nociceptive pain disorder.

In some embodiments, there is provided the use of a pharmaceutical composition described herein, for the treatment of osteoarthritis.

In some embodiments, there is provided the use of a pharmaceutical composition described herein, for the treatment of tendinitis.

In some embodiments, there is provided the use of a pharmaceutical composition described herein, for the treatment of chronic tendinitis.

In some embodiments, there is provided the use of a pharmaceutical composition described herein, for the treatment of pelvic pain.

In some embodiments, there is provided the use of a pharmaceutical composition described herein, for the treatment of neuropathic pain.

In some embodiments, there is provided the use of a pharmaceutical composition described herein, for the treatment of peripheral neuropathy.

In some embodiments, there is provided the use of a pharmaceutical composition described herein, for the treatment of PHN.

In some embodiments, there is provided the use of a pharmaceutical composition described herein, for the treatment of GERD.

In some embodiments, there is provided the use of a pharmaceutical composition described herein, for the treatment of diabetes.

In some embodiments, there is provided the use of a pharmaceutical composition described herein, for the treatment of obesity.

In some embodiments, there is provided the use of a pharmaceutical composition described herein, for the treatment of chronic cough.

In some embodiments, there is provided the use of a pharmaceutical composition described herein, for the treatment of COPD.

In some embodiments, there is provided the use of a pharmaceutical composition described herein, for the treatment of IBS.

In some embodiments, there is provided the use of a pharmaceutical composition described herein, for the treatment of overactive bladder.

In some embodiments, there is provided the use of a pharmaceutical composition described herein, for inhibiting VR1.

In some embodiments, there is provided a method for the treatment of a nociceptive pain disorder, comprising administering a pharmaceutical composition described herein to a subject in need thereof.

In some embodiments, there is provided a method for the treatment of a chronic nociceptive pain disorder, comprising administering a pharmaceutical composition described herein to a subject in need thereof.

In some embodiments, there is provided a method for the treatment of osteoarthritis, comprising administering a pharmaceutical composition described herein to a subject in need thereof.

In some embodiments, there is provided a method for the treatment of tendinitis, comprising administering a pharmaceutical composition described herein to a subject in need thereof.

In some embodiments, there is provided a method for the treatment of chronic tendinitis, comprising administering a pharmaceutical composition described herein to a subject in need thereof.

In some embodiments, there is provided a method for the treatment of pelvic pain, comprising administering a pharmaceutical composition described herein to a subject in need thereof.

In some embodiments, there is provided a method for the treatment of neuropathic pain, comprising administering a pharmaceutical composition described herein to a subject in need thereof.

In some embodiments, there is provided a method for the treatment of peripheral neuropathy, comprising administering a pharmaceutical composition described herein to a subject in need thereof.

In some embodiments, there is provided a method for the treatment of PHN, comprising administering a pharmaceutical composition described herein to a subject in need thereof.

In some embodiments, there is provided a method for the treatment of GERD, comprising administering a pharmaceutical composition described herein to a subject in need thereof.

In some embodiments, there is provided a method for the treatment of diabetes, comprising administering a pharmaceutical composition described herein to a subject in need thereof.

In some embodiments, there is provided a method for the treatment of obesity, comprising administering a pharmaceutical composition described herein to a subject in need thereof.

In some embodiments, there is provided a method for the treatment of chronic cough, comprising administering a pharmaceutical composition described herein to a subject in need thereof.

In some embodiments, there is provided a method for the treatment of COPD, comprising administering a pharmaceutical composition described herein to a subject in need thereof.

In some embodiments, there is provided a method for the treatment of IBS, comprising administering a pharmaceutical composition described herein to a subject in need thereof.

In some embodiments, there is provided a method for the treatment of overactive bladder, comprising administering a pharmaceutical composition described herein to a subject in need thereof.

In some embodiments, there is provided a method for inhibiting VR1, comprising administering a pharmaceutical composition described herein to a subject in need thereof.

In one aspect, there is provided a process for preparing a compound described herein, comprising:
treating a salt of (S)-2-(7-Cyano-1H-benzimidazol-1-yl)-N-{1-[4-(1-cyano-1-methylethyl)phenyl]ethyl}acetamide with a base, to obtain a free base compound; and
crystallizing the free base compound to obtain the compound of Formula I.

In some embodiments, the salt is (S)-2-(7-Cyano-1H-benzimidazol-1-yl)-N-{1-[4-(1-cyano-1-methylethyl)phenyl]ethyl}acetamide hydrochloride.

In some embodiments, the base is selected from the group consisting of sodium bicarbonate, potassium bicarbonate, cesium bicarbonate, sodium carbonate, potassium carbonate and cesium carbonate.

In some embodiments, the base is sodium bicarbonate.

In some embodiments, the base is solubilized in water.

In some embodiments, the salt of (S)-2-(7-Cyano-1H-benzimidazol-1-yl)-N-{1-[4-(1-cyano-1-methylethyl)phenyl]ethyl}acetamide is solubilized in a solvent selected from the group consisting of methanol, ethanol, water and mixtures thereof.

In some embodiments, the solvent is a mixture of methanol and water.

In some embodiments, the solvent is methanol.

In some embodiments, crystallizing the free base compound comprises:
solubilizing the free base compound by heating to obtain a free base solution;
cooling the free base solution to room temperature to obtain a free base slurry; and
filtering the free base slurry to obtain the compound of Formula I.

In some embodiments, the free base is solubilized in a mixture of water and methanol.

In some embodiments, filtering the free base slurry comprises:
obtaining a filter cake of the free base; and
drying the filter cake to obtain the compound of Formula I.

In some embodiments, drying the filter cake comprises drying the filter cake under vacuum at a temperature between about 40° C. and 45° C.

In one aspect, there is provided a process for preparing a compound described herein, comprising: subjecting (S)-2-(7-Cyano-1H-benzimidazol-1-yl)-N-{1-[4-(1-cyano-1-methylethyl)phenyl]ethyl}acetamide to solid-vapor diffusion of a solvent selected from the group consisting of water, ethanol, isopropyl alcohol (IPA), methyl isobutyl ketone (MIBK), ethyl acetate (EtOAc), isopropyl acetate (IPAc), Methyl tert-butyl ether (MTBE), tetrahydrofuran (THF) and toluene.

In one aspect, there is provided a process for preparing a compound described herein, comprising vapor diffusing MTBE into a solution of (S)-2-(7-Cyano-1H-benzimidazol-1-yl)-N-{1-[4-(1-cyano-1-methylethyl)phenyl]ethyl}acetamide in PA or IPAc, and subsequently cooling or evaporating the solution.

In one aspect, there is provided a process for preparing a compound described herein, comprising vapor diffusing n-heptane into a solution of (S)-2-(7-Cyano-1H-benzimidazol-1-yl)-N-{1-[4-(1-cyano-1-methylethyl)phenyl]ethyl}acetamide in methyl ethyl ketone (MEK).

In one aspect, there is provided a process for preparing a compound described herein, comprising agitating at room temperature a slurry of (S)-2-(7-Cyano-1H-benzimidazol-1-yl)-N-{1-[4-(1-cyano-1-methylethyl)phenyl]ethyl}acetamide in a solvent or solvent mixture selected from the group consisting of H₂O, EtOH, IPA, toluene, IPAc, EtOH/H₂O, EtOAc/n-heptane, MIBK/n-heptane, EtOH/MTBE, CHCl₃/MTBE and 2-MeTHF/toluene.

In one aspect, there is provided a process for preparing a compound described herein, comprising agitating at 50° C. a slurry of (S)-2-(7-Cyano-1H-benzimidazol-1-yl)-N-{1-[4-(1-cyano-1-methylethyl)phenyl]ethyl}acetamide in a solvent or solvent mixture selected from the group consisting of H₂O, IPA, toluene, IPAc, MTBE, EtOAc/n-heptane, MIBK/n-heptane, EtOH/MTBE, CHCl₃/MTBE and 2-MeTHF/toluene.

In one aspect, there is provided a process for preparing a compound described herein, comprising evaporating a solution of (S)-2-(7-Cyano-1H-benzimidazol-1-yl)-N-{1-[4-(1-cyano-1-methylethyl)phenyl]ethyl}acetamide in a solvent selected from the group consisting of MeOH, EtOH, IPA and acetone.

In one aspect, there is provided a process for preparing a compound described herein, comprising:
solubilizing (S)-2-(7-Cyano-1H-benzimidazol-1-yl)-N-{1-[4-(1-cyano-1-methylethyl)phenyl]ethyl}acetamide in methanol or ethanol; and
adding to the solution a polymer blend comprising polyvinyl pyrrolidone (PVP), polyvinyl alcohol (PVA), polyvinylchloride (PVC), polyvinyl acetate (PVAC), hypromellose (HPMC) and methyl cellulose (MC).

In one aspect, there is provided a process for preparing a compound described herein, comprising:
solubilizing (S)-2-(7-Cyano-1H-benzimidazol-1-yl)-N-{1-[4-(1-cyano-1-methylethyl)phenyl]ethyl}acetamide in methanol or ethanol; and
adding to the solution a polymer blend consisting of polyvinyl pyrrolidone (PVP), polyvinyl alcohol (PVA), polyvinylchloride (PVC), polyvinyl acetate (PVAC), hypromellose (HPMC) and methyl cellulose (MC).

In some embodiments, the weight ratio of the polymer blend is of 1:1:1:1:1:1.

In one aspect, there is provided a process for preparing a compound described herein, comprising:
solubilizing (S)-2-(7-Cyano-1H-benzimidazol-1-yl)-N-{1-[4-(1-cyano-1-methylethyl)phenyl]ethyl}acetamide in IPA or ethanol; and
adding to the solution a polymer blend comprising polycaprolactone (PCL), polyethylene glycol (PEG), poly (methyl methacrylate) (PMMA), sodium alginate (SA) and hydroxyethyl cellulose (HEC).

In one aspect, there is provided a process for preparing a compound described herein, comprising:
solubilizing (S)-2-(7-Cyano-1H-benzimidazol-1-yl)-N-{1-[4-(1-cyano-1-methylethyl)phenyl]ethyl}acetamide in IPA or ethanol; and
adding to the solution a polymer blend consisting of polycaprolactone (PCL), polyethylene glycol (PEG), poly (methyl methacrylate) (PMMA), sodium alginate (SA) and hydroxyethyl cellulose (HEC).

In some embodiments, the weight ratio of the polymer blend is of 1:1:1:1:1.

In one aspect, there is provided a process for preparing a compound described herein, comprising cooling a solution of (S)-2-(7-Cyano-1H-benzimidazol-1-yl)-N-{1-[4-(1-cyano-1-methylethyl)phenyl]ethyl}acetamide in a solvent or solvent mixture selected from the group consisting of IPA, toluene, MTBE, EtOH/n-heptane and CHCl₃/MTBE.

In one aspect, there is provided a process for preparing a compound described herein, comprising adding water to a solution of (S)-2-(7-Cyano-1H-benzimidazol-1-yl)-N-{1-[4-(1-cyano-1-methylethyl)phenyl]ethyl}acetamide in acetonitrile.

In one aspect, there is provided a process for preparing a compound described herein, comprising adding MTBE to a solution of (S)-2-(7-Cyano-1H-benzimidazol-1-yl)-N-{1-[4-(1-cyano-1-methylethyl)phenyl]ethyl}acetamide in ethanol or 2-MeTHF.

In one aspect, there is provided a process for preparing a compound described herein, comprising adding n-heptane to a solution of (S)-2-(7-Cyano-1H-benzimidazol-1-yl)-N-{1-[4-(1-cyano-1-methylethyl)phenyl]ethyl}acetamide in ethanol, MIBK or EtOAc.

In one aspect, there is provided a process for preparing a compound described herein, comprising adding toluene to a solution of (S)-2-(7-Cyano-1H-benzimidazol-1-yl)-N-{1-[4-(1-cyano-1-methylethyl)phenyl]ethyl}acetamide in 2-MeTHF.

In one aspect, there is provided a process for preparing a compound described herein, comprising adding water to a solution of (S)-2-(7-Cyano-1H-benzimidazol-1-yl)-N-{1-[4-(1-cyano-1-methylethyl)phenyl]ethyl}acetamide in DMSO.

In one aspect, there is provided a process for preparing a compound described herein, comprising heating a crystalline Form of (S)-2-(7-Cyano-1H-benzimidazol-1-yl)-N-{1-[4-(1-cyano-1-methylethyl)phenyl]ethyl}acetamide that exhibits an XRPD pattern having characteristic peaks expressed in degrees 2Θ (±0.2° 2Θ) at 4.77, 12.61, 14.05, 14.41, 16.68 and 17.06.

In one aspect, there is provided a process for preparing a compound described herein, comprising heating a crystalline Form of (S)-2-(7-Cyano-1H-benzimidazol-1-yl)-N-{1-[4-(1-cyano-1-methylethyl)phenyl]ethyl}acetamide that has a DSC thermogram exhibiting two endotherms having respective peak temperatures at about 135.1° C. and 163.2° C., and an exotherm having a peak temperature at about 137.1° C.

In some embodiments, the heating is performed up to at least 140° C., under nitrogen.

In one aspect, there is provided a process for preparing a compound described herein, comprising heating to at least 155° C., under nitrogen, a crystalline Form of
(S)-2-(7-Cyano-1H-benzimidazol-1-yl)-N-{1-[4-(1-cyano-1-methylethyl)phenyl]ethyl}acetamide that exhibits an XRPD pattern having characteristic peaks expressed in degrees 2Θ (±0.2° 2Θ) at 3.86, 4.52, 6.97, 12.44, 13.50 and 13.81.

In one aspect, there is provided a process for preparing a compound described herein, comprising heating to at least 155° C., under nitrogen, a crystalline Form of
(S)-2-(7-Cyano-1H-benzimidazol-1-yl)-N-{1-[4-(1-cyano-1-methylethyl)phenyl]ethyl}acetamide that has a DSC thermogram exhibiting three endotherms having respective peak temperatures at about 131.4° C., 152.7° C. and 164.3° C.

In one aspect, there is provided a process for preparing a compound described herein, comprising:
providing a slurry of a crystalline Form of (S)-2-(7-Cyano-1H-benzimidazol-1-yl)-N-{1-[4-(1-cyano-1-methylethyl)phenyl]ethyl}acetamide that exhibits an XRPD pattern having characteristic peaks expressed in degrees 2Θ (±0.2° 2Θ) at 4.24, 4.92, 8.15, 8.44, 8.73, 11.98 and 15.31, in IPA; and
agitating the slurry at room temperature.

In one aspect, there is provided a process for preparing a compound described herein, comprising:
providing a slurry of a crystalline Form of (S)-2-(7-Cyano-1H-benzimidazol-1-yl)-N-{1-[4-(1-cyano-1-methylethyl)phenyl]ethyl}acetamide that has a DSC thermogram exhibiting four endotherms that have peak temperatures at about 107.5° C., 122.6° C., 147.6° C. and 165.5° C., and two exotherms having peak temperatures at about 124.7° C. and 151.4° C., in IPA; and
agitating the slurry at room temperature.

In one aspect, there is provided a process for preparing a compound described herein, comprising:
providing a slurry of a crystalline Form of (S)-2-(7-Cyano-1H-benzimidazol-1-yl)-N-{1-[4-(1-cyano-1-methylethyl)phenyl]ethyl}acetamide that exhibits an XRPD pattern having characteristic peaks expressed in degrees 2Θ (±0.2° 2Θ) at 11.67, 13.09, 13.48, 14.06, 14.70 and 15.56, in IPA; and
agitating the slurry at room temperature.

In one aspect, there is provided a process for preparing a compound described herein, comprising:
providing a slurry of a crystalline Form of (S)-2-(7-Cyano-1H-benzimidazol-1-yl)-N-{1-[4-(1-cyano-1-methylethyl)phenyl]ethyl}acetamide that has a DSC thermogram exhibiting a first endotherm at a peak temperature of about 153.0° C. and a second endotherm at a peak temperature of about 162.6° C., in IPA; and
agitating the slurry at room temperature.

### BRIEF DESCRIPTION OF THE FIGURES

FIG. 1 is an X-ray powder diffractogram (XRPD) of Form A.

FIG. 2 is an XRPD of Form B.

FIG. 3 is an XRPD of Form C.

FIG. 4 is an XRPD of Form D.

FIG. 5 is an XRPD of Form E.

FIG. 6 is an XRPD of Form F.

FIG. 7 includes a differential scanning calorimetry (DSC) analysis of Form A in a hermetically-sealed pan at a scan rate of 10° C./minute under a nitrogen purge, and a thermogravimetric (TGA) analysis of Form A at a scan rate of 10° C./minute under a nitrogen purge.

FIG. 8 includes a DSC analysis of Form B in a hermetically-sealed pan at a scan rate of 10° C./minute under a nitrogen purge, and a TGA analysis of Form B at a scan rate of 10° C./minute under a nitrogen purge.

FIG. 9 includes a DSC analysis of Form C in a hermetically-sealed pan at a scan rate of 10° C./minute under a nitrogen purge, and a TGA analysis of Form C at a scan rate of 10° C./minute under a nitrogen purge.

FIG. 10 includes a DSC analysis of Form D in a hermetically-sealed pan at a scan rate of 10° C./minute under a nitrogen purge, and a TGA analysis of Form D at a scan rate of 10° C./minute under a nitrogen purge.

FIG. 11 includes a DSC analysis of Form E in a hermetically-sealed pan at a scan rate of 10° C./minute under a nitrogen purge, and a TGA analysis of Form E at a scan rate of 10° C./minute under a nitrogen purge.

FIG. 12 includes a DSC analysis of Form F in a hermetically-sealed pan at a scan rate of 10° C./minute under a nitrogen purge, and a TGA analysis of Form F at a scan rate of 10° C./minute under a nitrogen purge.

FIG. 13 is a series of variable temperature XRPDs of Form B changing to Form A upon heating.

FIG. 14 is a series of variable temperature XRPDs of Form C.

FIG. 15 is a series of variable temperature XRPDs of a mixture of Form C and Form F, changing to a mixture of Form A and Form F upon heating.

FIG. 16 is a comparison of XRPDs of Form D before and after vacuum drying.

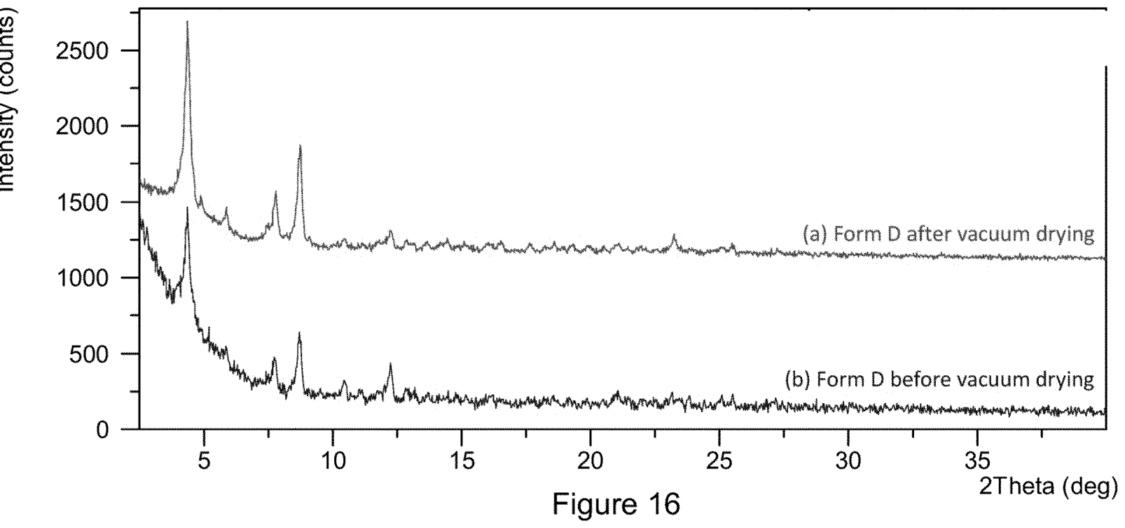

FIG. 17 is a series of variable temperature XRPDs of Form D changing to Form E upon heating to 80° C.

FIG. 18 is a series of variable temperature XRPDs of Form D changing to an amorphous form upon heating to 110° C., and further transforming to Form F upon heating to 124° C.

FIG. 19 is a series of XRPDs showing the slurry conversion of a mixture of Form A with Form E or Form F to Form A, after stirring at room temperature for 48 h.

FIG. 20 is a chart showing the inter-conversion relationships between the crystalline Forms.

FIG. 21 is a series of XRPDs of Form A, recorded at various temperatures, relative humidity and compression conditions.

### DETAILED DESCRIPTION

### Definitions

The term “stable”, as used herein, includes chemical stability and/or solid-state stability. A compound is considered chemically stable when the compound can be stored in an isolated solid form, or in the form of a solid formulation in which it may be provided in admixture with pharmaceutically acceptable carriers, diluents or adjuvants, under normal storage conditions, without any significant degree of chemical degradation or decomposition.

A compound is considered to have solid-state stability when the compound can be stored in an isolated solid form, or in the form of a solid formulation in which it may be provided in admixture with pharmaceutically acceptable carriers, diluents or adjuvants, under normal storage conditions, without any significant degree of solid state transformation (e.g. crystallisation, recrystallisation, loss of crystallinity, solid state phase transition, hydration, dehydration, solvatisation or desolvatisation).

Crystalline forms of solid chemical compounds influence not only their dissolution behavior (i.e. bioavailability) but also their solid-state stability. One way of comparing the solid-state stability of crystalline forms is to evaluate the relative “thermodynamic stability” of the crystalline forms. To evaluate the thermodynamic stability of crystalline forms, typical techniques include, but are not limited to, slurrying, slow evaporation, slow cooling, slow antisolvent addition, or a combination of these methods. Calorimetry techniques (e.g., Differential Scanning Calorimetry) can also be used to measure thermal events and phase transitions across a wide temperature range, and a comparison between the crystalline forms can give an indication as to their relative thermodynamic stability.

The expression “pharmaceutically acceptable carrier or excipient”, as used herein, includes without limitation any adjuvant, carrier, glidant, sweetening agent, diluent, preservative, dye/colorant, flavor enhancer, surfactant, wetting agent, dispersing agent, suspending agent, stabilizer, isotonic agent, solvent or emulsifier which is known as being acceptable for pharmaceutical use in humans or domestic animals.

The expression “pharmaceutical composition”, as used herein, refers to the formulation of a compound and a pharmaceutically acceptable carrier or excipient.

The term “about”, as used herein, generally means within an acceptable standard error of the mean, when considered by a person skilled in the art. For example, depending on the value or range considered, the term “about” can mean within 10%, within 5%, or within 1% of the value or range.

As used herein, the term “hydrate” refers to a crystalline form of a molecule that further comprises molecules of water incorporated into the crystalline lattice structure. The water molecules in the hydrate may be present in a regular arrangement and/or a non-ordered arrangement. The hydrate may comprise either a stoichiometric or nonstoichiometric amount of the water molecules. For example, a hydrate with a nonstoichiometric amount of water molecules may result from partial loss of water from the hydrate.

As used herein, the terms “anhydrate” or “anhydrous” refer to a crystalline form of a molecule per se that does not further comprise molecules of water incorporated into the crystalline lattice structure.

As used herein, the term “solvate” refers to a crystalline form of a molecule that further comprises molecules of a solvent or solvents incorporated into the crystalline lattice structure. The solvent molecules in the solvate may be present in a regular arrangement and/or a non-ordered arrangement. The solvate may comprise either a stoichiometric or nonstoichiometric amount of the solvent molecules. For example, a solvate with a nonstoichiometric amount of solvent molecules may result from partial loss of solvent from the solvate. The solvent can include various organic solvents. It should also be understood that a “solvate” can include a single solvent, a mixture of solvents or a mixture of a solvent (or solvents) and water.

The term “substantially the same”, used herein to describe X-ray diffraction patterns, is meant to include patterns in which peaks are within a standard deviation of ±0.2° 2Θ or an X-ray diffraction pattern comprising least 4, 5, 6, 7, 8, 9, 10, 11, 12, 13, 14, 15 or 16 peaks in common with the referenced pattern. Further, a person skilled in the art will appreciate that relative peak intensities will show inter-apparatus variability as well as variability due to degree of crystallinity, preferred orientation, prepared sample surface, and other factors. As such, the relative peak intensities should be taken as a qualitative measure.

The term “substantially pure”, when used in reference to a crystalline form of the compound of Formula (I), is meant to include a crystalline form which has a purity that is greater than about 90%. This means that the crystalline form may not contain more than about 10% of any other compound, and in particular, does not contain more than about 10% of any other crystalline form of the compound of Formula (I). Preferably, the term “substantially pure” means a crystalline form which has a purity that is greater than about 95%. This means that the crystalline form may not contain more than about 5% of any other compound, and in particular, does not contain more than about 5% of any other crystalline form of the compound of Formula (I). More preferably, the term “substantially pure” means a crystalline form which has a purity that is greater than about 99%. This means that the crystalline form may not contain more than about 1% of any other compound, and in particular, does not contain more than about 1% of any other crystalline form of the compound of Formula (I).

The term “solid” or “solid mixture” when used in reference to the compound of Formula (I), refers to a mixture of crystalline forms. For example, a solid or solid mixture can include at least two different crystalline forms of the compound of Formula (I). For example, a solid mixture can include crystalline Form A and one or more additional crystalline form(s) such as Form B, Form C, Form D, Form E and/or Form F.

XRPD data were obtained using PANalytical X-ray powder diffractometers, used in reflection mode. The radiation used was Cu Kα (λ=1.5418 Å). It should be understood that the 2Θ values listed herein are dependent on the type of radiation used, and that a person skilled in the art would understand that the XRPD of a given crystalline form will exhibit different 2Θ values if a different radiation is used (e.g., a molybdenum radiation).

The term “optically pure”, as used herein, refers to compounds which include a proportion of the desired enantiomer that is greater than that of the other enantiomer. An optically pure compound is generally made up of at least about 90%, 95% or 99% of the desired enantiomer, based upon 100 wt % total weight of the compound.

As used herein the terms “crystalline Form” or “polymorph” refers to crystal structure of a compound, having the same chemical composition but different spatial arrangements of the molecules, atoms, and/or ions forming the crystal structure.

Six crystalline Forms are obtained from polymorph screening of Compound 1, including three anhydrates (Form A, Form E and Form F), and three solvates or hydrates (Form B, Form C and Form D). Various crystalline Forms can convert to other crystalline Forms, as will be described in detail herein.

### Form A

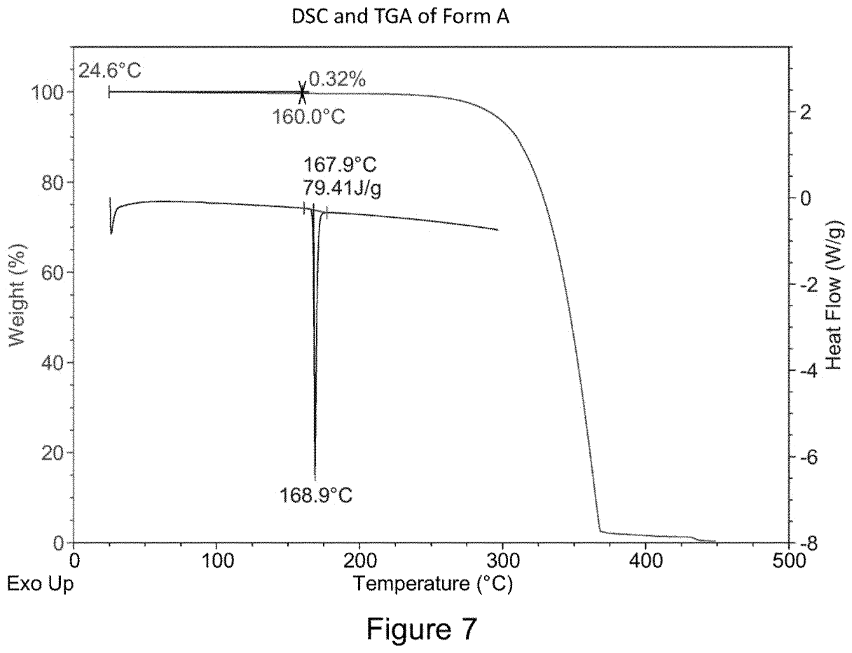

Crystalline Form A of Compound 1 is an anhydrate and is stable at room temperature. According to differential scanning calorimetry (DSC), Form A has a single endotherm, corresponding to melting, that has an onset at about 167.9° C. and a peak at about 168.9° C. The Thermogravimetric (TGA) analysis suggests that Form A is an anhydrate. The TGA analysis shows no substantial weight loss prior to decomposition starting at about 250° C. The DSC and TGA analyses of Form A are shown at FIG. 7.

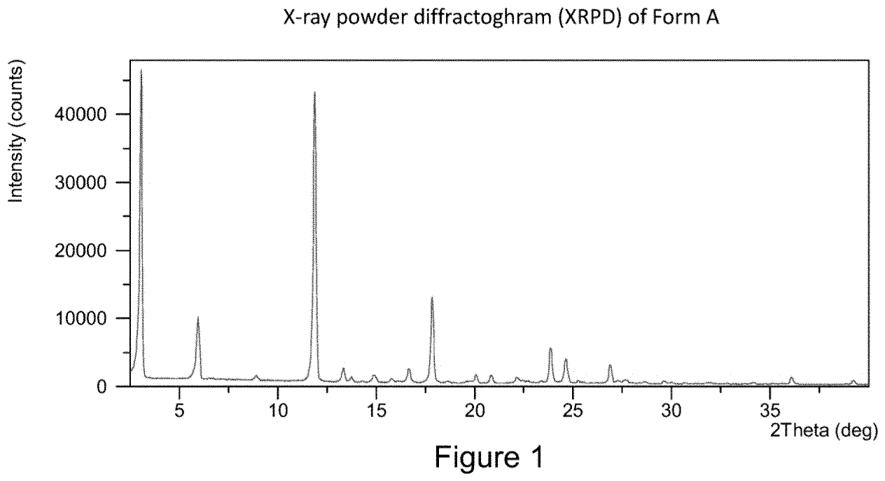

Form A of Compound 1 has an XRPD pattern substantially the same to that shown at FIG. 1. Peak locations and intensities for the XRPD pattern in FIG. 1 are provided in Table 1 below.

TABLE 1
Characteristic XRPD peaks (expressed in degrees
2θ ± 0.2° 2θ) and Relative
Intensities of Diffraction Lines for Form A of Compound 1

| Degrees 2θ (±0.2° 2θ) | I/I₁ |
|---|---|
| 3.07 | 100.00 |
| 5.96 | 21.18 |
| 8.92 | 2.83 |
| 11.89 | 92.23 |
| 13.35 | 5.31 |
| 13.75 | 2.33 |
| 14.90 | 3.04 |
| 16.67 | 5.05 |
| 17.85 | 27.85 |
| 20.08 | 3.05 |
| 20.84 | 2.97 |
| 22.15 | 2.31 |
| 23.86 | 11.75 |
| 24.63 | 8.12 |
| 26.88 | 6.33 |
| 39.20 | 2.24 |

Form A can be prepared by solid-vapor diffusion of a solvent selected from the group consisting of water, ethanol, isopropyl alcohol (IPA), methyl isobutyl ketone (MIBK), ethyl acetate (EtOAc), isopropyl acetate (IPAc), Methyl tert-butyl ether (MTBE), tetrahydrofuran (THF) and toluene.

Form A can also be prepared by liquid-vapor diffusion using IPA as the solvent and MTBE as the anti-solvent. A clear solution obtained after vapor diffusion of MTBE into a solution of Compound 1 in IPA was subjected to cooling to 5° C. or evaporation at room temperature to obtain crystals of Form A.

Form A can also be prepared by liquid-vapor diffusion using IPAc as the solvent and MTBE as the anti-solvent. A clear solution obtained after vapor diffusion of MTBE into a solution of Compound 1 in IPAc was subjected to cooling to 5° C. or evaporation at room temperature to obtain crystals of Form A.

Form A can also be prepared by liquid-vapor diffusion using methyl ethyl ketone (MEK) as the solvent and n-heptane as the anti-solvent.

Form A can also be prepared by preparing and agitating a slurry of Compound 1 at room temperature in a solvent or solvent mixture selected from the group consisting of H₂O, EtOH, IPA, toluene, IPAc, EtOH/H₂O, EtOAc/n-heptane, MIBK/n-heptane, EtOH/MTBE, CHCl₃/MTBE and 2-MeTHF/toluene. For example, the solvent mixtures can have the following ratios (v:v): EtOH/H₂O (704:296), EtOAc/n-heptane (1:1), MIBK/n-heptane (1:1), EtOH/MTBE (1:4), CHCl₃/MTBE (1:4) and 2-MeTHF/toluene (1:4).

Form A can also be prepared by preparing and agitating a slurry of Compound 1 at 50° C. in a solvent or solvent mixture selected from the group consisting of H₂O, IPA, toluene, IPAc, MTBE, EtOAc/n-heptane, MIBK/n-heptane, EtOH/MTBE, CHCl₃/MTBE and 2-MeTHF/toluene. For example, the solvent mixtures can have the following ratios (v:v): EtOAc/n-heptane (1:5), MIBK/n-heptane (1:5), EtOH/MTBE (1:9), CHCl₃/MTBE (1:9) and 2-MeTHF/toluene (1:9).

Form A can also be prepared by slow evaporation of a solution of Compound 1 in a solvent selected from the group consisting of MeOH, EtOH, IPA and acetone.

Form A can also be prepared by polymer-induced crystallization in a multiphase polymer system. In one example, Form A can be obtained by solubilizing Compound 1 in a solvent selected from the group consisting of MeOH and EtOH, and adding to the solution a polymer blend consisting of polyvinyl pyrrolidone (PVP), polyvinyl alcohol (PVA), polyvinylchloride (PVC), polyvinyl acetate (PVAC), hydromellose (HPMC), and methyl cellulose (MC) in a weight ratio of 1:1:1:1:1:1. In another example, Form A can be obtained by solubilizing Compound 1 in a solvent selected from the group consisting of IPA and EtOH, and adding to the solution a polymer blend consisting of polycaprolactone (PCL), polyethylene glycol (PEG), poly (methyl methacrylate) (PMMA), sodium alginate (SA), and hydroxyethyl cellulose (HEC) in a weight ratio of 1:1:1:1:1.

Form A can also be prepared by slow cooling of a solution of Compound 1 in a solvent or solvent mixture selected from the group consisting of IPA, toluene, MTBE, EtOH/n-heptane and CHCl₃/MTBE. For example, the solvent mixtures can have the following ratios (v:v): EtOH/n-heptane (1:4) and CHCl₃/MTBE (1:4).

Form A can also be prepared by addition of an anti-solvent to a solution of Compound 1 in a solvent. In one example, Form A can be prepared by adding water to a solution of Compound 1 in acetonitrile. In another example, Form A can be prepared by adding MTBE to a solution of Compound 1 in EtOH. In yet another example, Form A can be prepared by adding n-heptane to a solution of Compound 1 in EtOH. In yet another example, Form A can be prepared by adding n-heptane to a solution of Compound 1 in MIBK. In yet another example, Form A can be prepared by adding MTBE to a solution of Compound 1 in 2-MeTHF. In yet another example, Form A can be prepared by adding toluene to a solution of Compound 1 in 2-MeTHF. In yet another example, Form A can be prepared by adding n-heptane to a solution of Compound 1 in EtOAc. In yet another example, Form A can be prepared by adding water to a solution of Compound 1 in DMSO.

Another method for preparing Form A includes treating a salt of Compound 1 with a base, to obtain a free base compound. The free base compound can be crystallized to obtain crystals of Form A. The salt of Compound 1 can for example be a hydrochloride salt. The base can be selected from the group consisting of sodium bicarbonate, potassium bicarbonate, cesium bicarbonate, sodium carbonate, potassium carbonate and cesium carbonate. Preferably, the base is sodium bicarbonate, and is solubilized in water. Prior to being treated with the base, the salt of Compound 1 can be solubilized in a solvent selected from the group consisting of methanol, ethanol, water and mixtures thereof. Preferably, the solvent is methanol or a mixture of methanol and water. In some scenarios, crystallizing the free base compound comprises solubilizing the free base compound by heating to obtain a free base solution; cooling the free base solution to room temperature to obtain a free base slurry; and filtering the free base slurry to obtain the crystals of Form A. For example, the free base can be solubilized in a mixture of water and methanol. Filtering the free base slurry can include obtaining a filter cake of the free base, and drying the filter cake to obtain crystals of Form A. Drying of the filter cake can be performed under vacuum, at a temperature between about 40° C. and 45° C. Preferably, crystals of Form A can be prepared by treating the hydrochloride salt of Compound 1 solubilized in methanol, with aqueous sodium bicarbonate to generate Compound 1 that can crystallize in situ to furnish crystals of Form A.

The crystals obtained by the aforementioned methods may be recovered by techniques known in the art, such as, for example, filtration.

### Form B

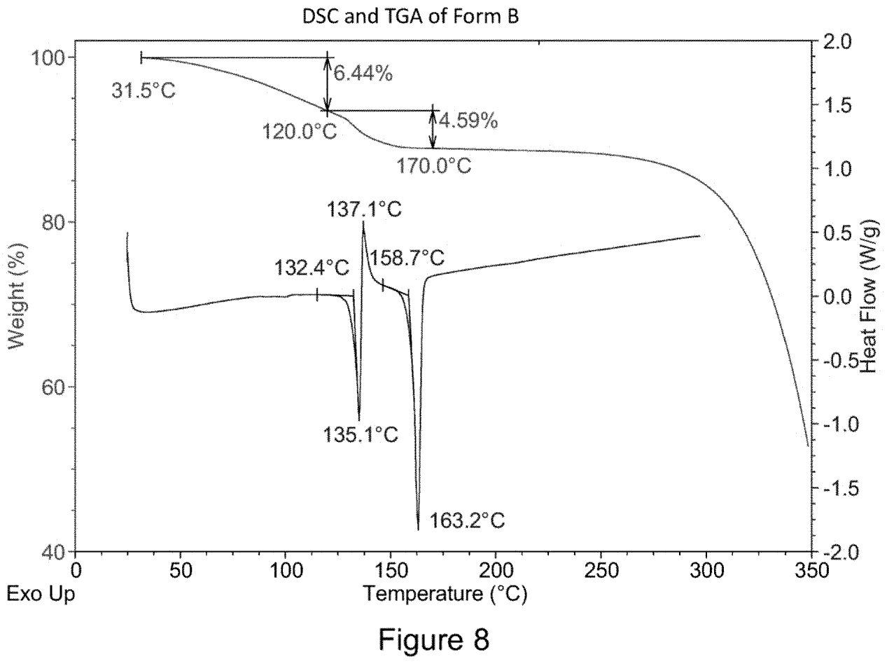

Crystalline Form B of Compound 1 is a solvate or hydrate. According to DSC, Form B has a first endotherm that has a peak at about 135.1° C., an exotherm that has a peak at about 137.1° C., and a second endotherm that has a peak at about 163.2° C. The TGA analysis shows a weight loss of about 6.4% between 31.5° C. and 120.0° C., and a further weight loss of about 4.6% between 120.0° C. and 170.0° C., suggesting that Form B is a solvate or hydrate. The TGA analysis shows no further substantial weight loss prior to decomposition that starts at about 250° C. The DSC and TGA analyses of Form B are shown at FIG. 8.

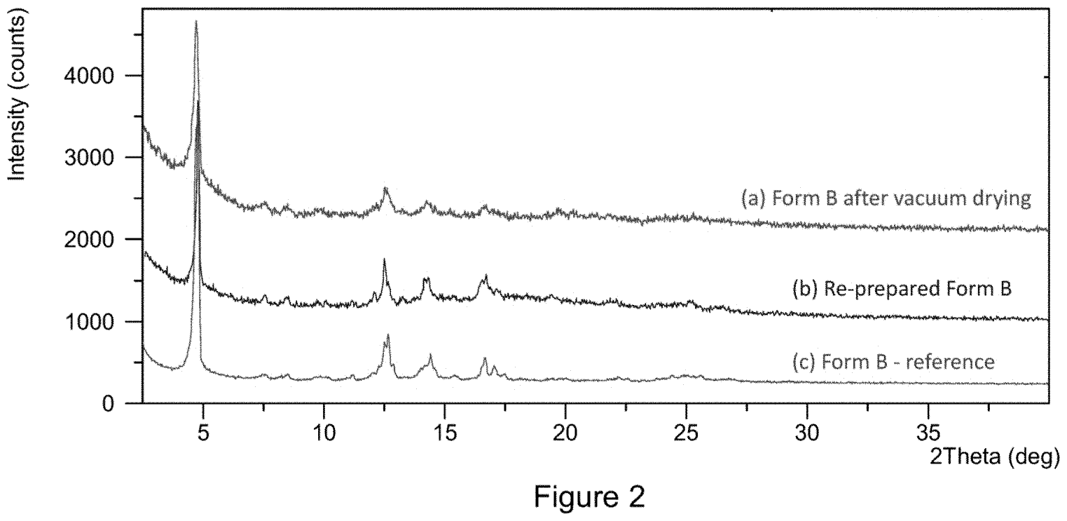

Form B of Compound 1 has an XRPD pattern substantially the same to that shown at FIG. 2(c). Peak locations and intensities for the XRPD pattern in FIG. 2(c) are provided in Table 2 below.

TABLE 2
Characteristic XRPD peaks (expressed in degrees
2Θ ± 0.2° 2Θ) and Relative
Intensities of Diffraction Lines for Form B of Compound 1

| Degrees 2Θ (±0.2° 2Θ) | I/I₁ |
|---|---|
| 4.77 | 100.00 |
| 12.61 | 14.57 |
| 14.05 | 4.75 |
| 14.41 | 9.75 |
| 16.68 | 9.36 |
| 17.06 | 5.85 |
| 17.48 | 2.66 |
| 24.43 | 2.42 |
| 25.60 | 2.61 |

Form B can be prepared by liquid-vapor diffusion using EtOH as the solvent and toluene as the anti-solvent. A clear solution obtained after vapor diffusion of toluene into a solution of Compound 1 in EtOH was subjected to cooling to 5° C. or evaporation at room temperature to obtain crystals of Form B.

Form B can also be prepared by liquid-vapor diffusion using dichloromethane (DCM) as the solvent and MTBE as the anti-solvent.

Form B can also be prepared by preparing and agitating a slurry of Compound 1 at room temperature in a solvent or solvent mixture selected from the group consisting of anisole and acetonitrile/toluene. For example, the solvent mixture can have the following ratio (v:v): acetonitrile/toluene (1:4).

Form B can also be prepared by preparing and agitating a slurry of Compound 1 at 50° C. in a solvent or solvent mixture selected from the group consisting of anisole and acetonitrile/toluene. For example, the solvent mixture can have the following ratio (v:v): acetonitrile/toluene (1:9).

Form B can also be prepared by slow evaporation of a solution of Compound 1 in a solvent selected from the group consisting of DCM and CHCl₃.

Form B can also be prepared by polymer-induced crystallization in a multiphase polymer system. In one example, Form B can be obtained by solubilizing Compound 1 in DCM, and adding to the solution a polymer blend consisting of polyvinyl pyrrolidone (PVP), polyvinyl alcohol (PVA), polyvinylchloride (PVC), polyvinyl acetate (PVAC), hypromellose (HPMC), and methyl cellulose (MC) in a weight ratio of 1:1:1:1:1:1. In another example, Form B can be obtained by solubilizing Compound 1 in CHCl₃, and adding to the solution a polymer blend consisting of polycaprolactone (PCL), polyethylene glycol (PEG), poly (methyl methacrylate) (PMMA), sodium alginate (SA), and hydroxyethyl cellulose (HEC) in a weight ratio of 1:1:1:1:1.

Form B can also be prepared by addition of an anti-solvent to a solution of Compound 1 in a solvent. In one example, Form B can be prepared by adding MTBE to a solution of Compound 1 in DCM.

The crystals obtained by the aforementioned methods may be recovered by techniques known in the art, such as, for example, filtration.

### Form C

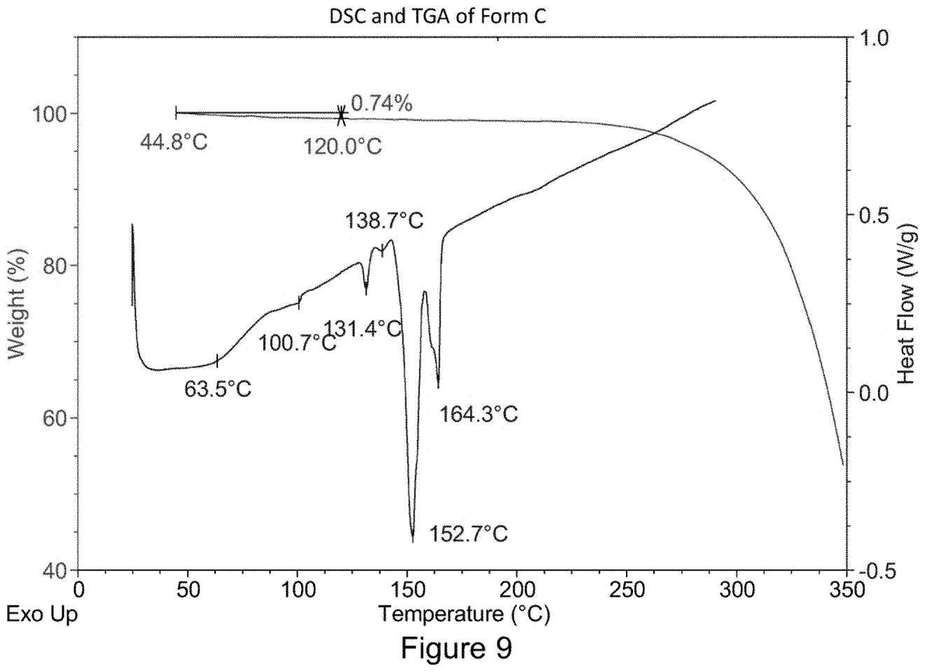

Crystalline Form C of Compound 1 is a solvate or hydrate. According to DSC, Form C has three main endotherms having peaks at about 131.4° C., 152.7° C. and 164.3° C. The TGA analysis suggests that Form C is a solvate or a hydrate. The TGA analysis shows no substantial weight loss prior to decomposition starting at about 250° C. The DSC and TGA analyses of Form C are shown at FIG. 9 and were performed after vacuum drying crystals of Form C.

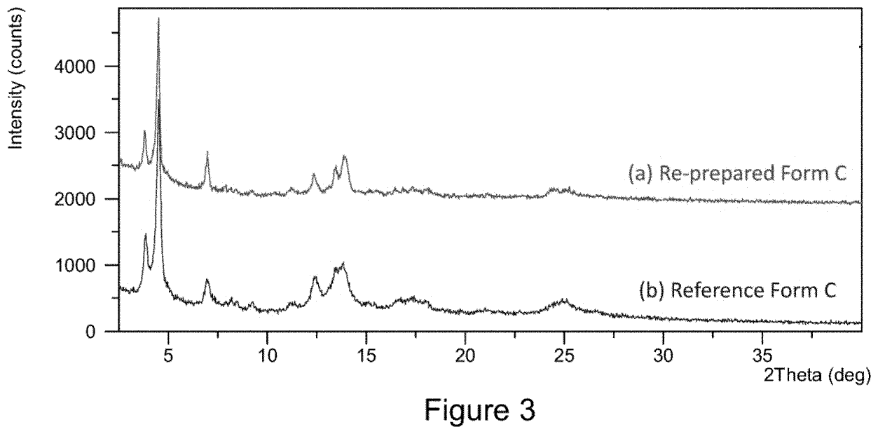

Form C of Compound 1 has an XRPD pattern substantially the same to that shown at FIG. 3(b). Peak locations and intensities for the XRPD pattern in FIG. 3(b) are provided in Table 3 below.

TABLE 3
Characteristic XRPD peaks (expressed in degrees
2Θ ± 0.2° 2Θ) and Relative
Intensities of Diffraction Lines for Form C of Compound 1

| Degrees 2Θ (±0.2° 2Θ) | I/I₁ |
|---|---|
| 3.86 | 29.73 |
| 4.52 | 100.00 |
| 6.97 | 11.30 |
| 8.29 | 2.00 |
| 9.23 | 3.05 |
| 11.27 | 3.47 |
| 12.44 | 17.47 |
| 13.50 | 21.31 |
| 13.81 | 23.38 |
| 16.69 | 5.92 |
| 17.31 | 6.81 |
| 18.07 | 4.87 |
| 24.99 | 5.36 |

Form C can be prepared by solid-vapor diffusion of a solvent selected from the group consisting of acetone, DCM and acetonitrile.

Form C can also be prepared by liquid-vapor diffusion using EtOAc as the solvent and n-heptane as the anti-solvent.

Form C can also be prepared by slow evaporation of a solution of Compound 1 in a solvent selected from the group consisting of EtOAc, IAPc, THF and acetonitrile. FIG. 3(a) shows the XRPD of re-prepared Form C obtained by slow evaporation of a solution of Compound 1 in EtOAc.

Form C can also be prepared by polymer-induced crystallization in a multiphase polymer system. In one example, Form C can be obtained by solubilizing Compound 1 in a solvent selected from the group consisting of acetone, THF and EtOAc, and adding to the solution a polymer blend consisting of polyvinyl pyrrolidone (PVP), polyvinyl alcohol (PVA), polyvinylchloride (PVC), polyvinyl acetate (PVAC), hypromellose (HPMC), and methyl cellulose (MC) in a weight ratio of 1:1:1:1:1:1. In another example, Form C can be obtained by solubilizing Compound 1 in a solvent selected from the group consisting of MEK and IPAc, and adding to the solution a polymer blend consisting of polycaprolactone (PCL), polyethylene glycol (PEG), poly (methyl methacrylate) (PMMA), sodium alginate (SA), and hydroxyethyl cellulose (HEC) in a weight ratio of 1:1:1:1:1.

Form C can also be prepared by slow cooling of a solution of Compound 1 in a solvent or solvent mixture selected from the group consisting of MIBK, EtOAc, IPAc and MEK/toluene. For example, the solvent mixture can have the following ratio (v:v): MEK/toluene (1:4). In some cases, such as when the solvent or solvent mixture is MIBK, EtOAc and MEK/toluene, a gel can be first obtained after cooling, and the gel then transform to Form C after slow evaporation at room temperature.

### Form D

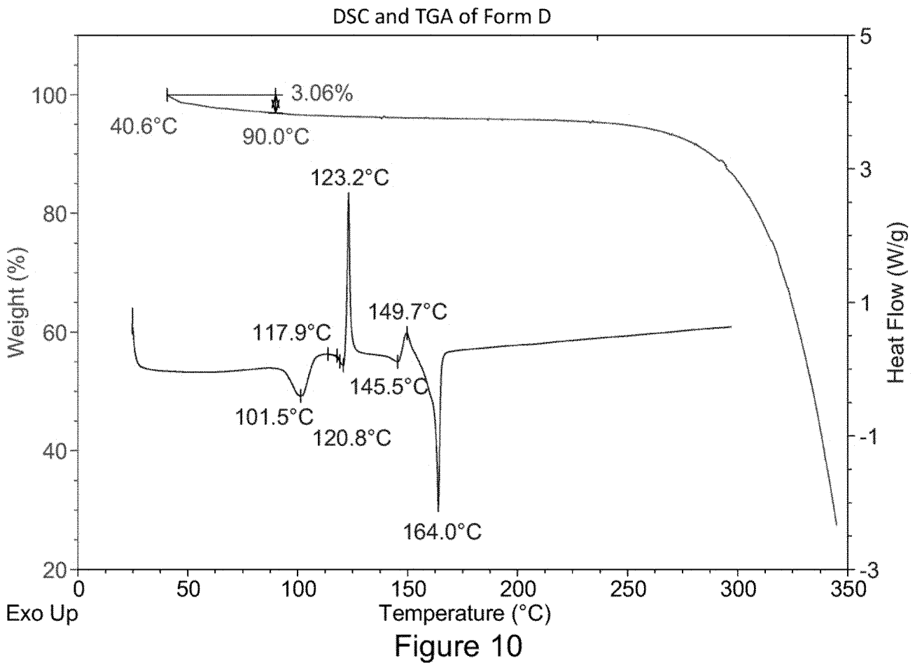

Crystalline Form D of Compound 1 is a solvate or hydrate. According to differential scanning calorimetry (DSC), Form D has multiple thermal events, including four endotherms having peaks at 101.5° C., 120.8° C., 145.5° C. and 164.0° C., as well as exotherms having peaks at 123.2° C. and 149.7° C. A first TGA analysis shows that Form D exhibits a weight loss of 3.1% between 40° C. and 90.0° C. and a second TGA analysis performed after drying under vacuum showed a weight loss of about 1.97% between 29.6° C. and 90.0° C. This suggests that Form D is a solvate or hydrate. The TGA analyses show no substantial weight loss prior to decomposition starting at about 250° C. The DSC and first TGA analyses (i.e., without drying under vacuum) of Form D are shown at FIG. 10.

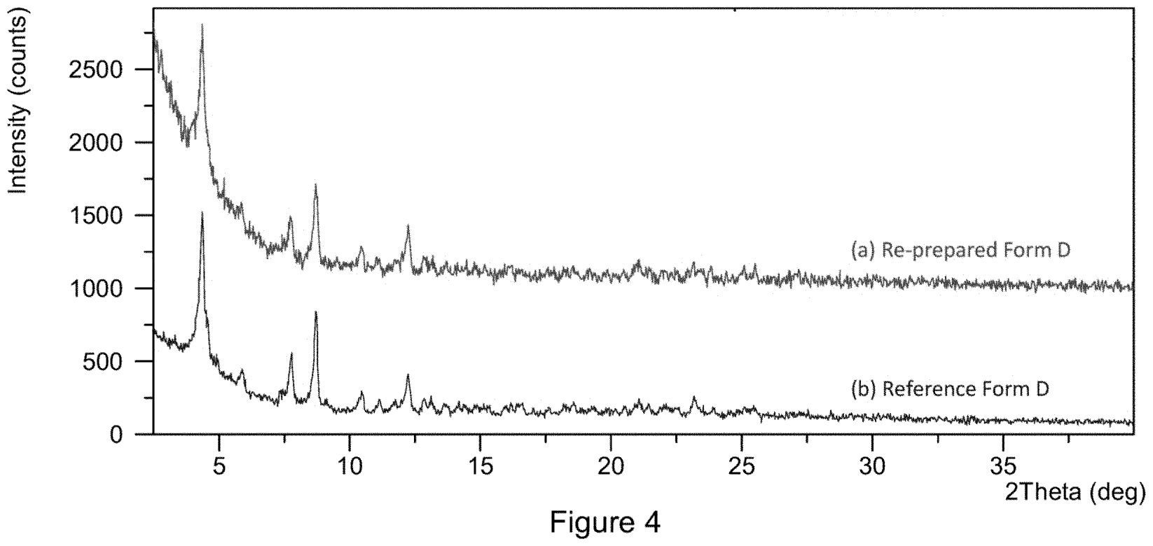

Form D of Compound 1 has an XRPD pattern substantially the same to that shown at FIG. 4(b). Peak locations and intensities for the XRPD pattern in FIG. 4(b) are provided in Table 4 below.

TABLE 4
Characteristic XRPD peaks (expressed in degrees
2Θ ± 0.2° 2Θ) and Relative
Intensities of Diffraction Lines for Form D of Compound 1

| Degrees 2Θ (±0.2° 2Θ) | I/I₁ |
|---|---|
| 4.38 | 100.00 |
| 5.87 | 12.14 |
| 7.78 | 37.50 |
| 8.73 | 72.17 |
| 10.47 | 15.15 |
| 11.16 | 8.13 |
| 11.75 | 6.64 |
| 12.26 | 32.16 |
| 12.89 | 10.17 |
| 13.12 | 11.31 |
| 13.65 | 7.61 |
| 14.16 | 7.77 |
| 15.13 | 5.88 |
| 16.60 | 10.17 |
| 17.64 | 5.04 |
| 18.26 | 7.69 |
| 18.61 | 11.81 |
| 19.32 | 6.61 |
| 19.93 | 5.67 |
| 20.53 | 8.92 |
| 21.08 | 14.78 |
| 21.46 | 8.04 |
| 22.16 | 9.30 |
| 22.56 | 8.35 |
| 23.21 | 16.59 |
| 23.93 | 6.28 |
| 25.12 | 7.51 |
| 25.49 | 9.76 |
| 26.88 | 4.71 |

Form D can be prepared by anti-solvent addition, by adding n-heptane to a solution of Compound 1 in DCM. FIG. 4(a) shows the XRPD of Form D re-prepared by anti-solvent addition of n-heptane to a solution of Compound 1 in DCM.

A mixture of Form D and Form A can be prepared by anti-solvent addition, by adding n-heptane to a solution of Compound 1 in 2-MeTHF.

A mixture of Form D and Form A can also be prepared by preparing and agitating a slurry of Compound 1 at room temperature in MTBE.

### Form E

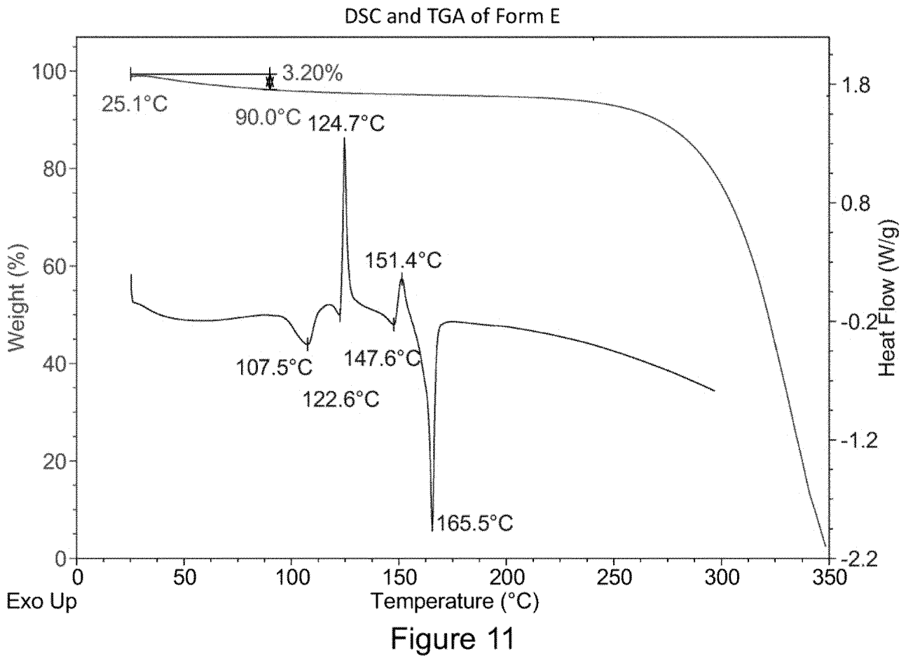

Crystalline Form E of Compound 1 is an anhydrate. According to DSC, Form E features multiple thermal events, including four endotherms that have peaks at about 107.5° C., 122.6° C., 147.6° C. and 165.5° C., as well as two exotherms having peaks at about 124.7° C. and 151.4° C. The TGA analysis suggests that Form E is an anhydrate. The TGA analysis shows a weight loss of 3.2% up to 90.0° C., and no substantial weight loss prior to decomposition starting at about 250° C. The DSC and TGA analyses of Form E are shown at FIG. 11.

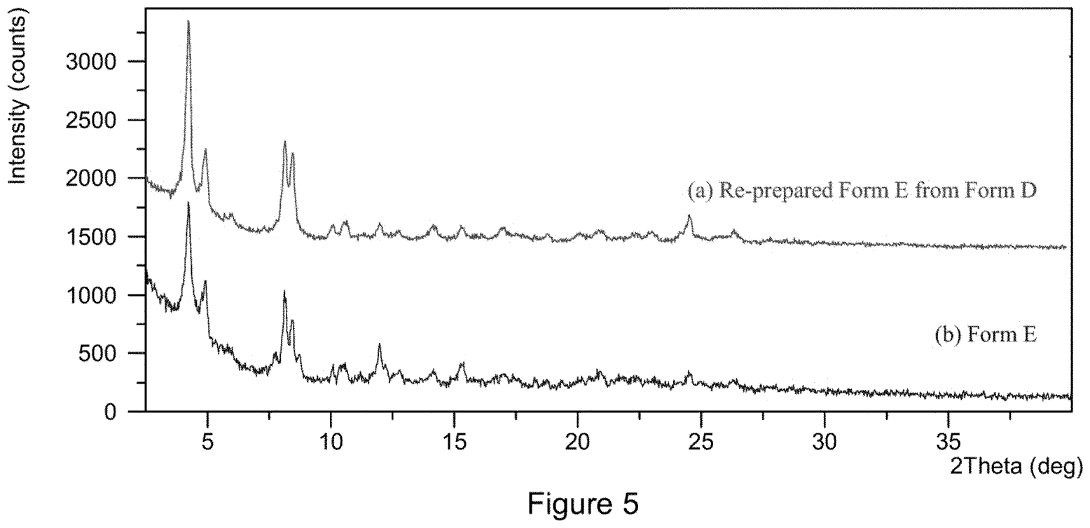

Form E of Compound 1 has an XRPD pattern substantially the same to that shown at FIG. 5(b). Peak locations and intensities for the XRPD pattern in FIG. 5(b) are provided in Table 5 below.

TABLE 5
Characteristic XRPD peaks (expressed in degrees
2Θ ± 0.2° 2Θ) and Relative
Intensities of Diffraction Lines for Form E of Compound 1

| Degrees 2Θ (±0.2° 2Θ) | I/I₁ |
|---|---|
| 4.24 | 100.00 |
| 4.92 | 46.97 |
| 5.88 | 5.42 |
| 7.76 | 16.73 |
| 8.15 | 65.30 |
| 8.44 | 48.04 |
| 8.73 | 19.74 |
| 10.08 | 10.55 |
| 10.60 | 11.28 |
| 11.98 | 30.14 |
| 12.78 | 8.99 |
| 14.15 | 9.21 |
| 15.31 | 15.21 |
| 16.99 | 8.16 |
| 20.88 | 7.42 |
| 24.51 | 10.71 |
| 26.22 | 5.15 |

Form E can be prepared by slow evaporation of a solution of Compound 1 in MEK.

The crystals obtained by the aforementioned method may be recovered by techniques known in the art, such as, for example, filtration.

### Form F

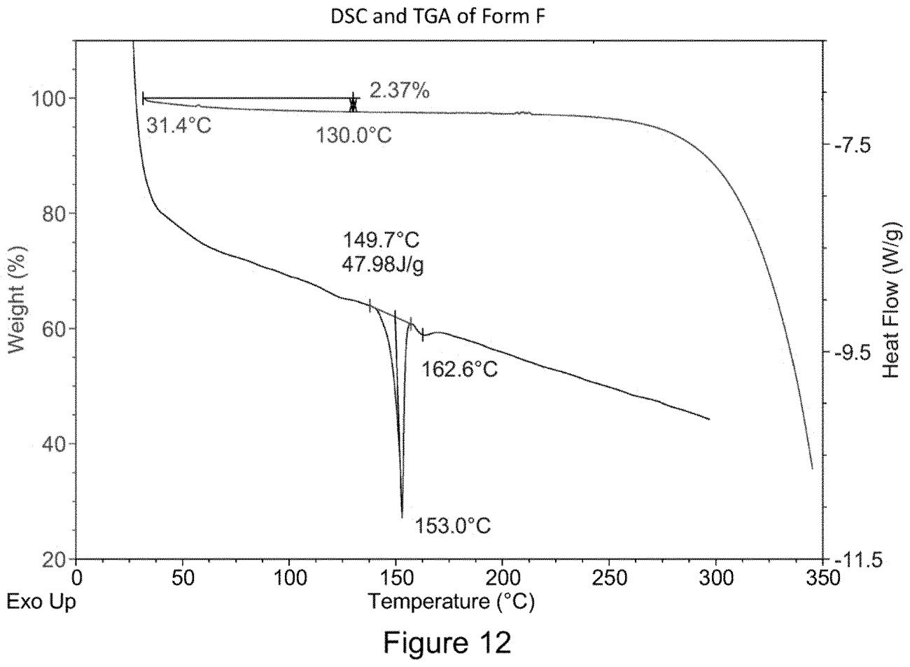

Crystalline Form F of Compound 1 is an anhydrate. According to DSC analysis, Form F has a first sharp endotherm having a peak at about 153.0° C. and a second smaller endotherm having a peak at about 162.6° C. The TGA shows a weight loss of 2.4% up to 130.0° C., and suggests that Form F is an anhydrate. The TGA analysis shows no substantial weight loss prior to decomposition starting at about 250° C. The DSC and TGA analyses of Form F are shown at FIG. 12.

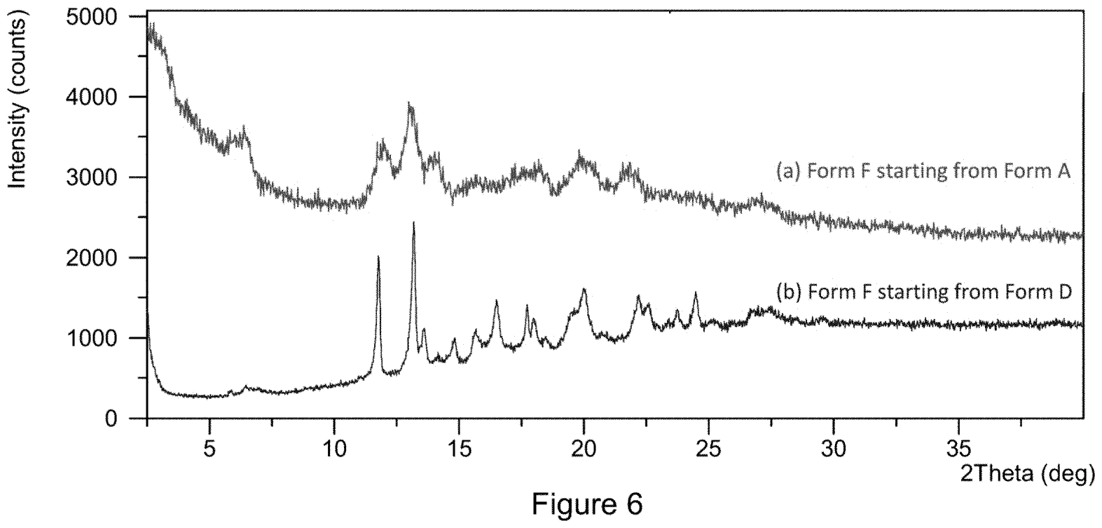

Form F of Compound 1 has an XRPD pattern substantially the same to that shown at FIG. 6. Peak locations and intensities for the XRPD pattern in FIG. 6(b) are provided in Table 6 below.

TABLE 6
Characteristic XRPD peaks (expressed in degrees
2Θ ± 0.2° 2Θ) and Relative
Intensities of Diffraction Lines for Form F of Compound 1

| Degrees 2Θ (±0.2° 2Θ) | I/I₁ |
|---|---|
| 5.75 | 2.68 |
| 6.35 | 5.17 |
| 11.67 | 81.09 |
| 13.09 | 100.00 |
| 13.48 | 29.38 |
| 14.06 | 11.30 |
| 14.70 | 20.30 |
| 15.56 | 23.74 |
| 16.40 | 42.76 |
| 17.62 | 36.73 |
| 17.88 | 28.64 |
| 18.36 | 15.07 |
| 19.35 | 27.06 |
| 19.91 | 44.24 |
| 22.08 | 35.57 |
| 22.47 | 28.96 |
| 23.27 | 15.89 |
| 23.63 | 23.78 |
| 24.38 | 33.25 |
| 25.06 | 13.33 |
| 26.66 | 16.40 |
| 27.35 | 16.86 |
| 29.47 | 6.49 |

Form F can be prepared starting from Form A or from Form 0, as will be described below.

Interconversion of Crystalline Forms

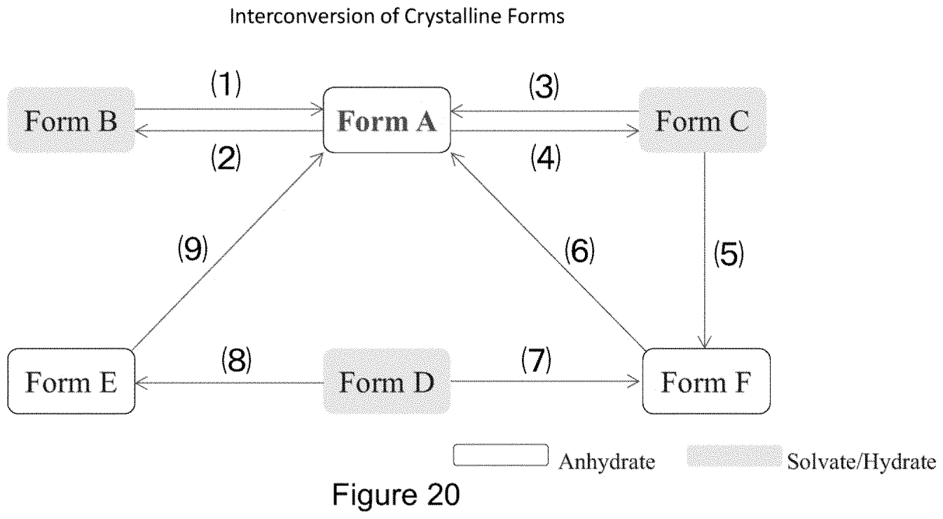

Now referring to FIG. 20, a chart showing the inter-conversion relationships between the crystalline Forms of Compound 1 is shown:

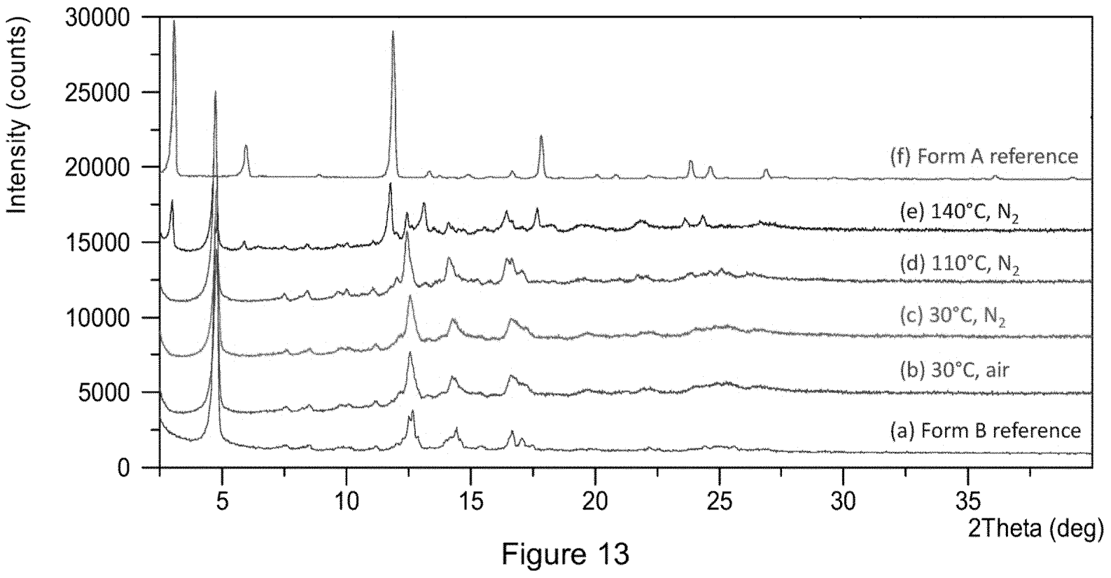

Crystals of Form B can be converted (1) to crystals of Form A. For example, crystals of Form B are converted to crystals of Form A by heating the crystals of Form B to 140° C. under nitrogen. A series of variable temperature XRPD of Form B changing to Form A upon heating is shown at FIG. 13.

Crystals of Form A can be converted (2) to crystals of Form B. For example, crystals of Form A are converted to crystals of Form B by preparing a slurry of crystals of Form A in anisole and agitating at room temperature, or by preparing a slurry of crystals of Form A in a mixture of acetonitrile/toluene and agitating at 50° C. FIG. 2(b) shows an XRPD of re-prepared Form B obtained from a slurry of Form A in anisole at 50° C.

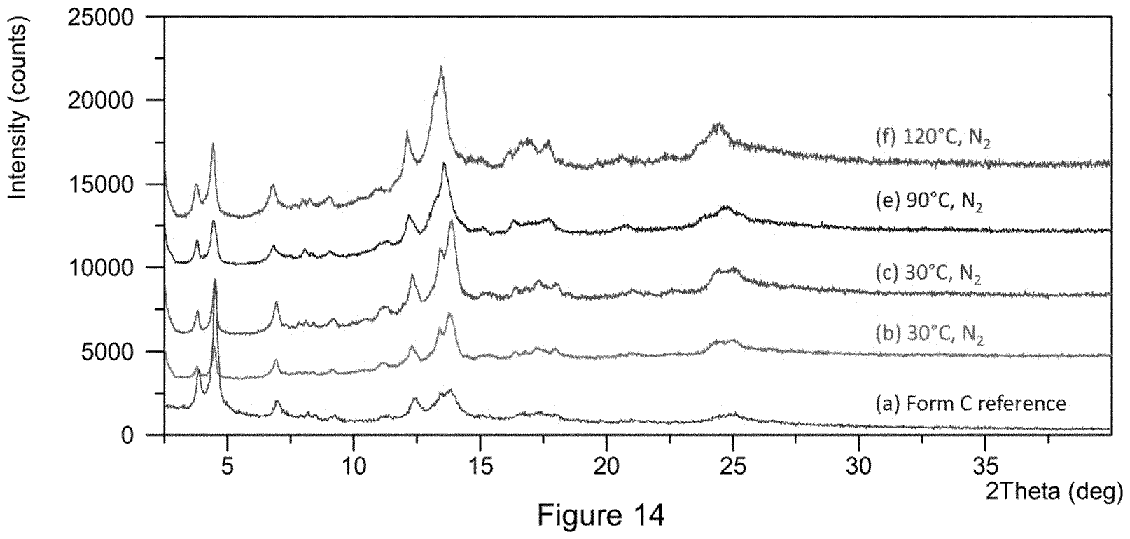

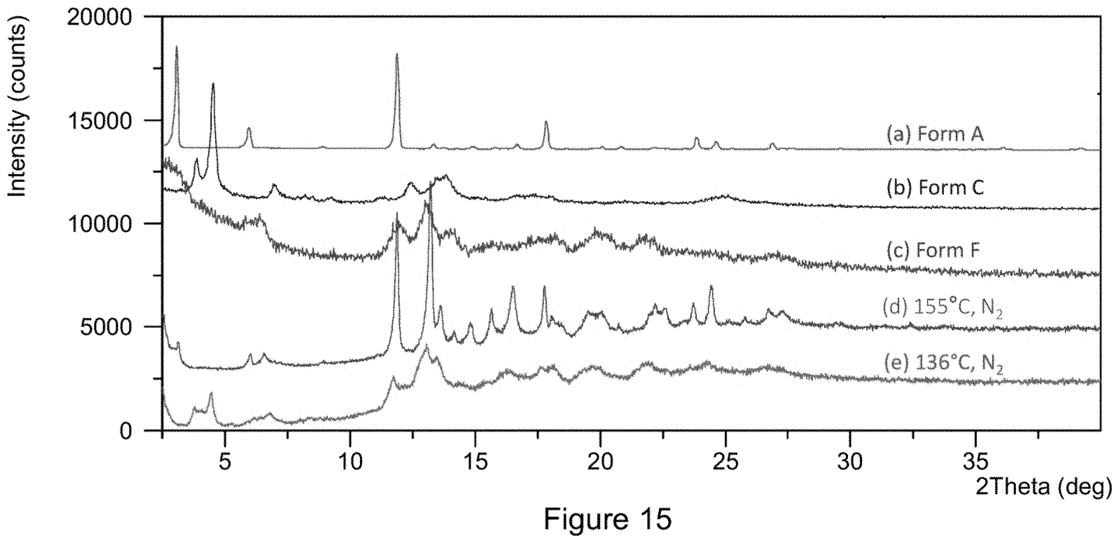

Crystals of Form C can be converted (3) to crystals of Form A. For example, crystals of Form C are converted to crystals of Form A by heating the crystals of Form C to 155° C. under nitrogen. Furthermore, crystals of Form C can be converted (5) to crystals of Form F. For example, crystals of Form C are converted to crystals of Form F by heating to 136° C. under nitrogen. A series of variable temperature XRPD of Form C are shown at FIG. 14, and shows that Form C is stable at least up to 120° C. As shown at FIG. 15, a series of variable temperature XRPD shows that a mixture of crystals of Form C and Form F is obtained after heating crystals of Form C to 136° C. Further heating of this sample generates a mixture of crystals of Form A and Form F.

Crystals of Form A can be converted (4) to crystals of Form C. For example, crystals of Form A are converted to crystals of Form C by solid-vapor diffusion using an acetone/DCM/acetonitrile solvent mixture.

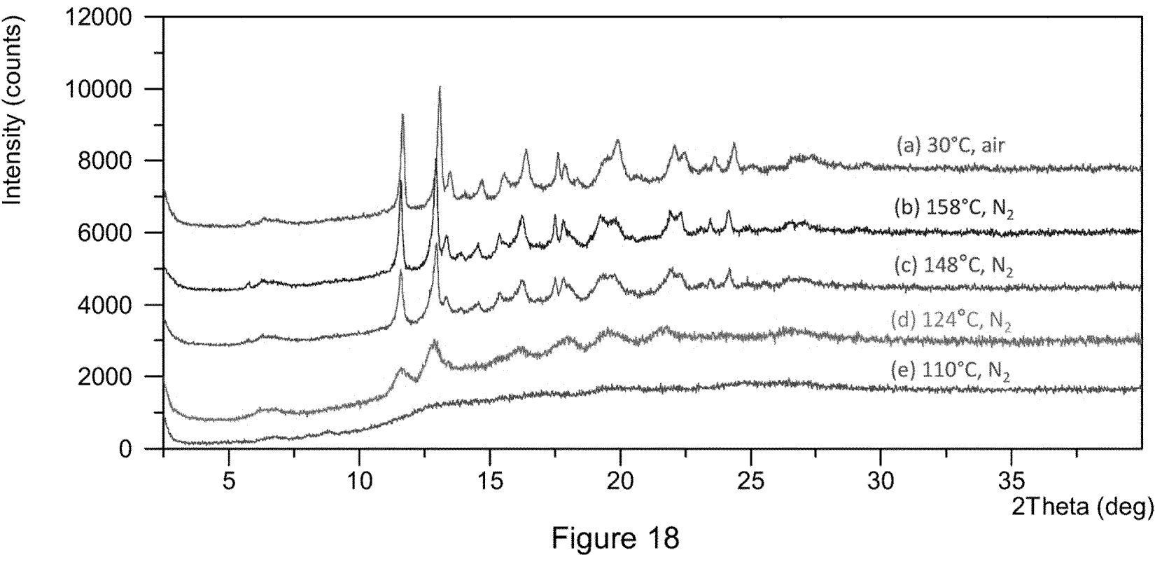

Crystals of Form D can be converted (7) to crystals of Form F. For example, crystals of Form D are converted to crystals of Form F by heating crystals of Form D to 124° C. under nitrogen. As shown at FIG. 18, Form D converts to an amorphous solid after heating to 110° C. Further heating this sample to 124° C. generates Form F. The variable temperature XRPD analyses also show that the crystallinity increases with temperature.

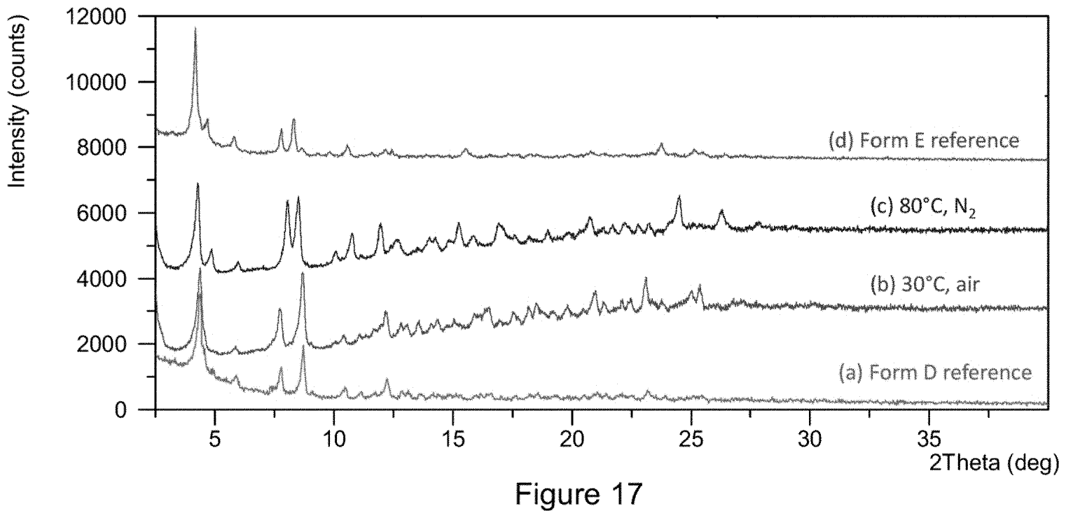

Crystals of Form D can be converted (8) to crystals of Form E. For example, crystals of Form D are converted to crystals of Form E by heating crystals of Form D to 80° C. under nitrogen, as shown at FIG. 17. FIG. 5(a) also shows an XRPD of a re-prepared Form E obtained by heating Form D to 80° C. under nitrogen.

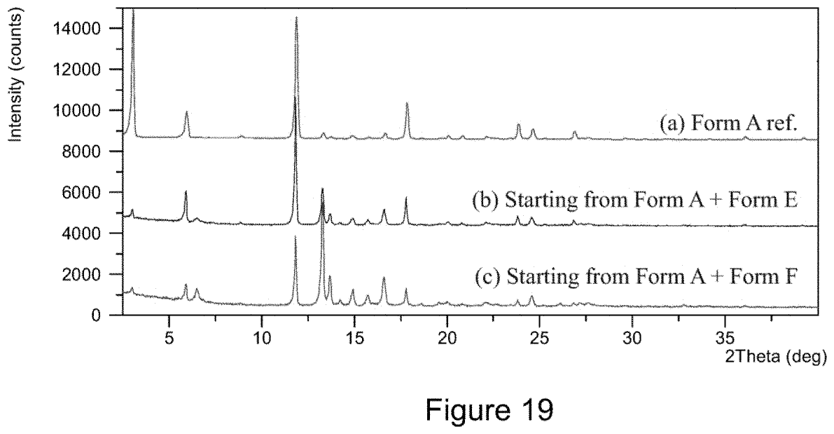

Crystals of Form F can be converted (6) to crystals of Form A. Similarly, crystals of Form E can be converted (9) to crystals of Form A. For example, a mixture of crystals of Form E and Form A is converted to crystals of Form A by slurry conversion of the mixture in IPA at room temperature. Similarly, a mixture of crystals of Form F and Form A is converted to crystals of Form A by slurry conversion of the mixture in IPA at room temperature. This suggests that Form A is the thermodynamically stable form at room temperature among the three anhydrates. FIG. 19 shows comparative XRPD of Form A, mixture of Form A and Form E and mixture of Form A and Form F.

Amorphous Form, Gel and low crystallinity Forms

Non-crystalline Forms (amorphous solid, gel-like material) and low crystallinity material can also be prepared.

In one example, a gel-like material can be prepared by liquid-vapor diffusion using MIBK as the solvent and toluene as the anti-solvent.

In another example, a gel-like material can be prepared by slow cooling of a solution of Compound 1 in anisole.

In another example, a gel-like material can be prepared by slow evaporation of a solution of Compound 1 in 2-MeTHF.

In another example, a gel-like material can be prepared by addition of MTBE as an anti-solvent, in a solution of Compound 1 in IPA.

In another example, an amorphous solid can be prepared by addition of toluene as an anti-solvent, in a solution of Compound 1 in acetonitrile. An amorphous solid can also be prepared by addition of toluene as an anti-solvent, in a solution of Compound 1 in ethanol. An amorphous solid can also be prepared by addition of MTBE as an anti-solvent, in a solution of Compound 1 in MIBK. An amorphous solid can also be prepared by addition of toluene as an anti-solvent, in a solution of Compound 1 in EtOAc.

In another example, a low crystallinity material can be prepared by addition of toluene as an anti-solvent, in a solution of Compound 1 in MIBK. A low crystallinity material can also be prepared by addition of toluene as an anti-solvent, in a solution of Compound 1 in DCM. A low crystallinity material can also be prepared by addition of MTBE as an anti-solvent, in a solution of Compound 1 in EtOAc.

In another example, an amorphous form can be obtained by heating crystals of Form A above melting temperature, yet under the decomposition temperature (e.g., 200° C.), and by subsequently cooling the melt to room temperature, for example at a rate of about 10° C./min under N₂ protection.

Form D can be converted to an amorphous Form by heating to 110° C.

### Solid-State Stability of the Crystalline Forms

Compound 1 appears to be chemically stable up to the decomposition temperature of about 250° C. However, some of the crystalline forms exhibit a greater solid-state stability compared to other crystalline forms. The solid-state stability of the crystalline forms is compared by evaluating the relative “thermodynamic stability” of the crystalline forms and the interconversion between crystalline forms upon heating or when slurrying.

The TGA analysis of Form B shows a partial weight loss starting at 120° C., suggesting that Form B features at least a partial loss of water or solvent molecules upon heating. It was also shown that Form B converts to Form A upon heating, suggesting that Form A is more thermodynamically stable than Form B.

According to DSC, Form C shows several endotherms prior to melting. Form C converts to Form F upon heating to 136° C. under nitrogen, and to Form A upon heating to 155° C. under nitrogen, suggesting that Forms A and F are more thermodynamically stable than Form C.

According to DSC, Form D shows several thermal events prior to melting. Form D converts to Form E upon heating to 80° C. under nitrogen and to Form F upon heating to 124° C. under nitrogen, suggesting that Forms E and F are more thermodynamically stable than Form D.

According to DSC, Form E shows several thermal events prior to melting, Form F shows a first sharp endotherm at about 153.0° C. and a second smaller endotherm having a peak at about 162.6° C. Form E and Form F can be converted to Form A by slurry conversion. The DSC analyses and slurrying observation suggest that Form A is more thermodynamically stable than Form E and Form F.

Form A is an anhydrate that is stable up to the melting point at about 167.9° C. Form A can be prepared by treating a hydrochloride salt of compound 1 with sodium bicarbonate to obtain a free base compound and directly crystallizing the free base compound. Form A can also be directly obtained from several other crystalline forms, namely from Forms B, C, E and F. Form A is also the crystalline form that appears to be obtainable via the most routes compared to all the other crystalline forms.

From the above, it therefore appears that Form A is more thermodynamically stable than all of the other crystalline forms identified herein, namely Forms B, C, D, E and F.

### Preparation of Compound 1

Compound 1 can be prepared, for example, according to steps 1 to 3 shown below. The experimental procedure for step 3 is described in Example 1. It should be understood that the reagents shown in the reaction schemes below can be substituted by other reagents of similar reactivity, as would be known by a person skilled in the art and to the extent that the reaction still proceeds as planned.

Precursors (S)-2-[4-(1-aminoethyl)phenyl)phenyl]-2-methylpropionitrile (S)-mandelic acid salt 11 and (7-cyano-1H-benzimidazo-1-yl)acetic acid 13 can be prepared, respectively, using Step 1 and Step 2 shown below.

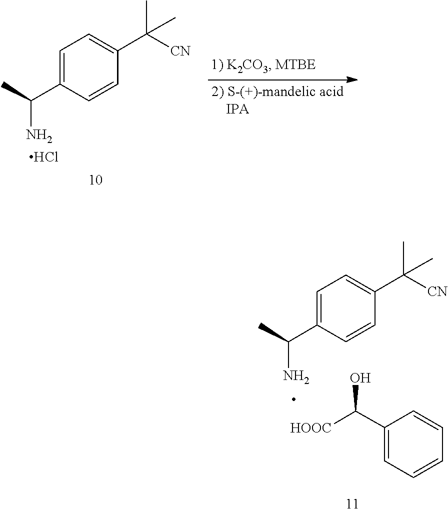

### Step 1: Purification of Precursor 11

In Step 1, 11 can be prepared from corresponding hydrochloride salt 10 using chiral resolution by salt exchange. The purpose of this salt exchange operation is to improve both the chemical and optical purity of the amine. The amine portion of 10 is liberated by reaction of 10 with an aqueous solution of potassium carbonate and can be extracted into MTBE. Subsequent exposure of the free amine to (S)-(+)-mandelic acid in refluxing 2-propanol can then furnish the corresponding mandelate salt.

It should be understood that other conditions and/or other reactants can be used to perform the chiral resolution by salt exchange. For example, the salt 10 can be a hydrobromide salt, a fumarate salt or any other suitable salt that would allow for the chiral resolution by salt exchange to occur. The base can be selected from the group consisting of potassium bicarbonate, sodium bicarbonate, potassium carbonate, sodium carbonate, or any other base that is able to deprotonate the salt 10. It should also be understood that the solvent used to extract the amine portion of 10 can be any organic solvent in which the free amine has a sufficient solubility. Non-limiting examples of solvents include dichloromethane and chloroform. Finally, it should also be understood that other types of chiral resolving agents can be used. Non-limiting examples of chiral resolving agents include optically pure tartaric acid, camphor-10-sulphonic acid, dibenzoyl tartaric acid and ditoluyl tartaric acid.

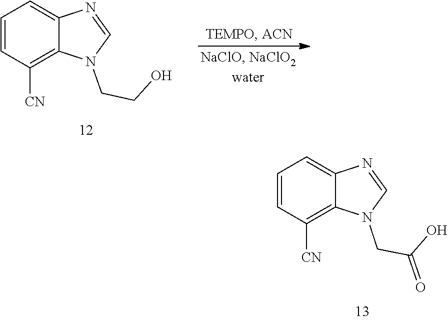 [SMILES: N#Cc1cccc2ncn(CCO)c12 ; N#Cc1cccc2ncn(CC(=O)O)c12]

### Step 2: Synthesis of Precursor 13

In Step 2, 1-(2-hydroxyethyl)-1H-benzimidazole-7-carbonitrile 12 can be transformed into (7-cyano-1H-benzimidazo-1-yl)acetic acid 13 via two oxidations that take place in tandem. The hydroxyl group in 12 can first be converted to the corresponding aldehyde, which can then undergo further reaction to form the carboxylic acid 13. It should be understood that other oxidation reagents can be used to convert alcohol 12 into carboxylic acid 13.

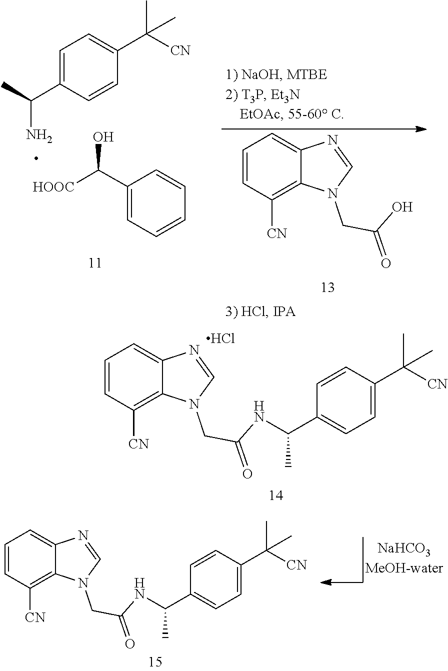

### Step 3: Synthesis of 15

The synthesis of Compound 1 (or 15) can be performed from (7-cyano-1H-benzimidazo-1-yl)acetic acid 13 and the amine derived from (S)-2-[4-(1-aminoethyl)phenyl)phenyl]-2-methylpropionitrile (S)-mandelic acid salt 11, as shown in Step 3.

The mandelate 11 can first be treated with sodium hydroxide to produce the corresponding free amine which can then be condensed with the carboxylic acid 13 using T3P as coupling agent, to furnish 15 as a crude product. It should be understood that other bases can be used to treat 11 and produce the free amine. For example, potassium hydroxide can be used for generating the free amine. Other coupling agents can also be used instead of T3P for condensing the free amine of 11 with the carboxylic acid 13. For example, EDC or DCC can be used as coupling agents.

The crude product 15 can be purified via the formation of a corresponding benzimidazolium salt and subsequent neutralization of the salt. For example, the corresponding benzimidazolium hydrochloride salt 14 can be obtained by adding hydrochloric acid to a mixture of the crude product 15 in isopropanol. Subsequent treatment of the benzimidazolium hydrochloride salt 14 in methanol with aqueous sodium bicarbonate can then regenerate 15 as a purified product.

In some scenarios, the purified 15 can then crystallize in situ to furnish the polymorph of Form A. Other conditions can allow obtaining the polymorph of Form A, as described herein.

### Methods, Uses, Formulation and Administration

### Methods and Uses

The compound of Formula (I) is an antagonist of vanilloid receptor 1 (VR-1).

The compound of Formula (I) may be used for the treatment of pain, acute pain, chronic pain, nociceptive pain, acute nociceptive pain, chronic nociceptive pain, neuropathic pain, acute neuropathic pain, chronic neuropathic pain, inflammatory pain, acute inflammatory pain, and/or chronic inflammatory pain.

The compound of Formula (I) may also be used for the treatment of osteoarthritis (such as of the knee), chronic tendinitis, pelvic pain, neuropathic pain and peripheral neuropathy (such as postherpetic neuralgia—PHN), gastroesophageal reflux disease (GERD), irritable bowel syndrome (IBS), diabetes, obesity, chronic cough, chronic obstructive pulmonary disease (COPD) and overactive bladder.

The compound of Formula (I) may also be used in the preparation of a medicament for the treatment of the above-described disorders in a warm-blooded animal, preferably a mammal, more preferably a human.

The treatment of such disorders may include administering to a warm-blooded animal, preferably a mammal, more preferably a human, in need of such treatment, an effective amount of a compound of Formula (I) or a pharmaceutically acceptable salt thereof.

### Formulations

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The term “patient or subject” as used herein refers to a mammal. A subject therefore refers to, for example, dogs, cats, horses, cows, pigs, guinea pigs, and the like. Preferably the subject is a human. When the subject is a human, the subject may be either a patient or a healthy human.

In some embodiments, the therapeutically effective amount of a compound as defined herein, or a pharmaceutically acceptable salt thereof, can be administered to a patient alone or admixed with a pharmaceutically acceptable carrier.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of the present description that, upon administration to a recipient, is capable of providing, either directly or indirectly, a compound of the present description or an inhibitory active metabolite or residue thereof.

Compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a provided compound, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming micro-encapsulated matrices of the compound in biodegradable polymers such as polylactide-polyglycolide. Depending upon the ratio of compound to polymer and the nature of the particular polymer employed, the rate of compound release can be controlled.

Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of the present description with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, lozenges, capsules, pastilles, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

Provided compounds can also be in micro-encapsulated form with one or more excipients as noted above. The solid dosage forms of tablets, dragees, lozenges, capsules, pastilles, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of the present description include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of the present description. Additionally, the description contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Pharmaceutically acceptable compositions provided herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promotors to enhance bioavailability, fluorocarbons, and/or other conventional solubilizing or dispersing agents.

Pharmaceutically acceptable compositions provided herein may be formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of provided compounds that may be combined with carrier materials to produce a composition in a single dosage form will vary depending upon the patient to be treated and the particular mode of administration. Provided compositions may be formulated such that a dosage of between 0.01-100 mg/kg body weight/day of the inhibitor can be administered to a patient receiving these compositions.

It should also be understood that a specific dosage and treatment regimen for any particular patient will depend upon a variety of factors, including age, body weight, general health, sex, diet, time of administration, rate of excretion, drug combination, the judgment of the treating physician, and the severity of the particular disease being treated. The amount of a provided compound in the composition will also depend upon the particular compound in the composition.

Compounds or compositions described herein may be administered using any amount and any route of administration effective for treating or lessening the severity of the disorders or diseases as contemplated herein. The exact amount required will vary from subject to subject, depending on the species, age, and general condition of the subject, the severity of the disorder or disease, the particular agent, its mode of administration, and the like. Provided compounds are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this disclosure can be administered to humans and other animals orally, rectally, parenterally, intracisternally, intravaginally, intraperitoneally, topically (as by powders, ointments, or drops), buccally, as an oral or nasal spray, or the like, depending on the severity of the infection being treated. In certain embodiments, provided compounds may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

### Combinations

Depending upon the particular condition, or disease, to be treated, additional therapeutic agents that are normally administered to treat that condition may also be present in the compositions of this disclosure or administered separately as a part of a dosage regimen. As used herein, additional therapeutic agents that are normally administered to treat a particular disease, or condition, are known as “appropriate for the disease, or condition, being treated.”

In some embodiments, the composition of a compound or compounds described herein can be in combination with an additional therapeutic agent.

It will be understood, however, that the total daily usage of the compounds and compositions of the present description will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The total daily dose of the compounds of the present description administered to a subject in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In one embodiment, treatment regimens according to the present description comprise administration to a patient in need of such treatment from about 10 mg to about 1000 mg of the compound(s) of the present description per day in single or multiple doses.

As used herein, the term “combination,” “combined,” and related terms refers to the simultaneous or sequential administration of therapeutic agents in accordance with the present description. For example, a provided compound may be administered with another therapeutic agent simultaneously or sequentially in separate unit dosage forms or together in a single unit dosage form. Accordingly, an embodiment of the present description provides a single unit dosage form comprising a provided compound, an additional therapeutic agent, and a pharmaceutically acceptable carrier, adjuvant, or vehicle for use in the methods of the present description.

The amount of both, a provided compound and additional therapeutic agent (in those compositions which comprise an additional therapeutic agent as described above) that may be combined with the carrier materials to produce a single dosage form will vary depending upon the host treated and the particular mode of administration. Preferably, compositions should be formulated such that a dosage of between 0.01-100 mg/kg body weight/day of a provided compound can be administered.

In those compositions which comprise an additional therapeutic agent, that additional therapeutic agent and the provided compound may act synergistically. Therefore, the amount of additional therapeutic agent in such compositions will be less than that required in a monotherapy utilizing only that therapeutic agent. In such compositions a dosage of between 0.01-1,000 g/kg body weight/day of the additional therapeutic agent can be administered.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compound of Formula (I) may be administered concurrently, simultaneously, sequentially or separately with another compound or compounds. Non-limiting examples of combination products may be selected from the following:
(i) neuropathic pain therapies including for example gabapentin, lidoderm, pregablin and equivalents including but not limited to a pharmaceutically acceptable salt and pharmaceutically active isomer(s) and metabolite(s) thereof.
(ii) nociceptive pain therapies including for example celecoxib, etoricoxib, lumiracoxib, rofecoxib, valdecoxib, diclofenac, loxoprofen, naproxen, paracetamol and equivalents including but not limited to a pharmaceutically acceptable salt and pharmaceutically active isomer(s) and metabolite(s) thereof.
(iii) urinary incontinence therapies including for example darifenacin, falvoxate, oxybutynin, propiverine, robalzotan, solifenacin, tispium, tolterodine and equivalents including but not limited to a pharmaceutically acceptable salt and pharmaceutically active isomer(s) and metabolite(s) thereof.

### EXPERIMENTS AND EXAMPLES

### Example 1: Synthesis of Form a of Compound 1—Experimental Procedure for Step 3

A process for preparing crystalline Form A of the compound of Formula (I): (S)-2-(7-Cyano-1H-benzimidazol-1-yl)-N-{1-[4-(1-cyano-1-methylethyl)phenyl]ethyl} acetamide (also referred to herein as 15) was performed.

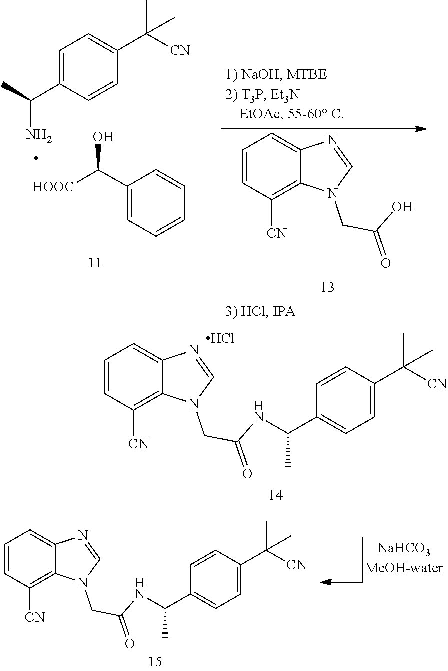

### Step 3: Synthesis of 15/Compound 1

A 500 mL 3-Neck round bottom flask equipped with a thermometer, mechanical stirrer, condenser and nitrogen inlet was charged with 20.71 g of 11 (60.8 mmol, 1.02 eq wrt 13) and 60.85 mL of a 2M NaOH (2.04 eq wrt 13) solution. The resulting yellow slurry was agitated for 15 minutes at 20-25° C. 120 mL of Methyl tert-butyl ether (MTBE, 10 parts wrt 13) were added in one portion and the resulting mixture was agitated for a minimum of 15 minutes to obtain a biphasic yellow solution. The biphasic yellow solution was separated using a separatory funnel and the upper organic layer was set aside. The aqueous layer (having a pH>10) was extracted with 60 mL MTBE (5 parts wrt 13). The lower aqueous layer was removed, and the upper organic layer was combined with the previously obtained organic layer.

The combined organic phase (about 180 mL) was transferred to a 500 mL 3-neck round bottom flask equipped with a thermometer, mechanical stirrer, an addition funnel and a nitrogen inlet. 180 mL of ethyl acetate (EtOAc, 15 parts wrt 13) were added and the resulting solution was heated to reflux for 1 hour under nitrogen. The solvent was removed by distillation through a distillation bridge under atmospheric pressure to about 108 mL (9 parts wrt 13), and the solution of free amine was subsequently cooled to 20-25° C.

A 1 L 3-neck round bottom flask equipped with a thermometer, mechanical stirrer and a nitrogen inlet was charged with 12.0 g of 13 (59.6 mmol, 1.0 eq) and 120 mL of EtOAc (5 parts wrt 13). The resultant slurry was agitated for 15 minutes at 20-25° C. The solution of free amine previously obtained was transferred to the 13 slurry, and the flask was rinsed with 24 mL of EtOAc (2 parts wrt 13), to obtain a suspension.

The suspension was charged with 23.7 g of a solution of T3P (propylphosphonic anhydride, 1.25 eq wrt 13) in EtOAc and rinsed with 24 mL of EtOAc (2 parts wrt 13). 12.6 mL of triethylamine (1.5 eq wrt 13) were added to the suspension and rinsed with 24 mL of EtOAc (2 parts wrt 13). The yellow slurry obtained was then heated to 55-60° C. under nitrogen with moderate agitation for 2.5 hours. The reaction mixture was then cooled to 20-25° C. and 120 mL of water (10 parts wrt 13) were then added. The resultant biphasic mixture was agitated for a minimum of 1 hour, and then transferred to a separatory funnel to remove the lower aqueous layer.

The upper organic layer was transferred back to the 1 L 3-neck round bottom flask equipped with a thermometer, mechanical stirrer and nitrogen inlet. 120 mL of a 2M aqueous NaOH solution (10 parts wrt 13) was added, and the resulting mixture was agitated moderately for a minimum of 1 hour. The biphasic mixture was transferred to a separatory funnel and the lower aqueous layer was removed. The organic layer was transferred back to the 1 L 3-neck round bottom flask, equipped with a thermometer, mechanical stirrer and nitrogen inlet. 120 mL of water (10 parts wrt 13) was added to the flask and the mixture was agitated for a minimum of 30 minutes. The biphasic mixture was transferred to a separatory funnel and the lower aqueous layer was removed. The organic layer was transferred back to the 1 L 3-neck round bottom flask equipped with a thermometer, mechanical stirrer and nitrogen inlet. 120 mL of water (10 pars wrt 13) was added to the flask and the resulting mixture was warmed to 35-40° C. and agitated for a minimum of 30 minutes. The biphasic mixture was separated in a separatory funnel while still warm. The organic layer was transferred to a 500 mL 3-neck round bottom flask equipped with a thermometer, mechanical stirrer and nitrogen inlet.

Under moderate agitation, 60 mL of EtOAc (5 parts wrt 13) were added and the resulting solution was heated to 55-60° C. 17.3 mL of a 3.8M solution of HCl (1.1 eq wrt 13) in isopropyl alcohol were added and the resulting mixture was heated to reflux. Solvent was distilled off under atmospheric pressure until a target volume of about 240 mL was reached. The temperature was lowered to 20-25° C. with moderate agitation, then further decreased to 0-5° C. and agitated for a minimum 1 h at 0-5° C. The slurry was suction filtered through a Buchner funnel using Whatman™ filter paper. The filter cake was washed with 2×36 mL EtOAc (2×3 parts wrt 13) and dried under suction. The filter cake was transferred to a petri dish and further dried in a vacuum oven for 18 hours at 40-4° C. to furnish the benzimidazolium hydrochloride salt 14 as a white to light beige solid.

10.0 g of 14 (24.5 mmol, 1.0 eq) and 60 mL MeOH (6 parts wrt 14) were added to a 250 mL 3-neck round bottom flask equipped with a thermometer, mechanical stirrer, condenser and a nitrogen inlet. The slurry was agitated for 10 minutes at 20-25° C. to form a solution. 1.0 g of activated charcoal DARCO™ KB-G (0.1 parts w/w wrt 14) suspended in 15 mL of MeOH (1.5 parts wrt 14) was added. The addition equipment was rinsed with 15 mL of MeOH, and the suspension obtained was agitated for a minimum of 1 h at 20-25° C. The suspension was then charged with 1.0 g of Celite™ (0.1 parts w/w wrt 14) suspended in 15 mL of MeOH, and the addition equipment was rinsed with 15 mL of MeOH. The resulting mixture was agitated for a minimum of 15 minutes at 20-25° C. The suspension was then suction filtered through a Buchner funnel using Whatman filter paper. The filter cake was washed with 2×20 mL of MeOH (2×2 parts wrt 14). The combined filtrate and washing was transferred to a 250 mL 3-neck round bottom flask equipped with a thermometer, mechanical stirrer, addition funnel and nitrogen inlet. The volume of the solution was reduced by atmospheric distillation through a distillation bridge to about 60-65 mL (6-6.5 parts wrt 14) at 65-67° C.

The solution obtained was cooled to 2-25° C. and then charged with a solution made up with 2.05 g of sodium bicarbonate (1 eq wrt 14) dissolved in 35 mL of water (3.5 parts wrt 14) over 30 minutes. The reaction mixture was heated to 40-45° C., and 15 mL of water (1.5 parts wrt 14) was charged to produce a white suspension. The suspension was charged with another 15 mL of water (1.5 parts wrt 14) at 40-45° C. The mixture was heated to reflux under moderate agitation to form a clear solution and kept for 5-10 minutes. The solution was cooled to 20-25° C. over a period of 1 h, and agitated for a minimum of 1 h at 20-25° C. The slurry was filtered through a Buchner funnel using Whatman filter paper under suction. The filter cake was washed with 2×30 mL of a mixture of MeOH—H₂O (2:3, v/v, 2×3 parts wrt 14). The cake was washed with 40 mL of water (4 parts wrt 14) and then was kept under suction with a nitrogen flow. The filter cake was transferred to a petri dish and dried further in a vacuum oven for 18 hours at 40-45° C. to afford the product 15 that crystallized in-situ as crystals of Form A having a rod-like structure. The overall yield of Step 3 was 77% and the purity of the crystals of Form A was evaluated at 99.89% by HPLC.

### Example 2: Solubility Experiments for Crystals of Form A

Experiments were conducted to evaluate the solubility of crystals of Form A at room temperature (25° C.±2° C.). Approximately 2 mg solids were added into a 3-mL glass vial. Solvents as listed in Table 7 were then added stepwise into the vials until the solids were dissolved or a total volume of 1 mL was reached. The results are summarized in Table 7, and were used to guide solvent selection in the polymorph screening.

TABLE 7
Approximate solubility of crystals of Form A at room temperature

| Solvent | Solubility (mg/mL) | Solvent | Solubility (mg/mL) |
|---|---|---|---|
| MeOH | S > 42.0 | 1,4-Dioxane | S > 36.0 |
| EtOH | S > 40.0 | DCM | S > 40.0 |
| IPA | 7.0 < S < 21.0 | CHCl₃ | S > 42.0 |
| Acetone | S > 38.0 | Toluene | S < 2.1 |
| MEK | S > 40.0 | Anisole | 7.3 < S < 22.0 |
| MIBK | 18.0 < S < 36.0 | DMAc | S > 36.0 |
| EtOAc | 22.0 < S < 44.0 | DMSO | S > 44.0 |
| IPAc | 6.7 < S < 20.0 | ACN | S > 40.0 |
| MTBE | S < 1.8 | n-Heptane | S < 2.2 |
| THF | S > 40.0 | H₂O | S < 2.1 |
| 2-MeTHF | S > 36.0 | — | — |

### Example 3: Solid-Vapor Diffusion

Solid-vapor diffusion experiments were conducted using 12 different solvents. For each experiment, approximately 15 mg of crystals of Form A were weighed and placed into a 3-mL vial. The 3-mL vial was placed into a 20-mL vial containing about 2 mL of a volatile solvent. The 20-mL vial was sealed with a cap and kept at room temperature for 2˜7 days allowing solvent vapor to interact with the sample. The solids were tested by XRPD, and the results summarized in Table 8 showed that crystals of Form A or Form C were obtained.

TABLE 8
Summary of solid-vapor diffusion experiments

| Entry | Solvent | Solid form |
|---|---|---|
| Sol-Vap #1 | H₂O | Form A |
| Sol-Vap #2 | EtOH | Form A |
| Sol-Vap #3 | IPA | Form A |
| Sol-Vap #4 | Acetone | Form C |
| Sol-Vap #5 | MIBK | Form A |
| Sol-Vap #6 | EtOAc | Form A |
| Sol-Vap #7 | IPAc | Form A |
| Sol-Vap #8 | MTBE | Form A |
| Sol-Vap #9 | THF | Form A |
| Sol-Vap #10 | DCM | Form C |
| Sol-Vap #11 | Toluene | Form A |
| Sol-Vap #12 | ACN | Form C |

### Example 4: Liquid-Vapor Diffusion

Seven liquid-vapor diffusion experiments were conducted. For each experiment, approximately 15 mg of crystals of Form A were weighed and placed into a 3-mL vial. The crystals were dissolved in a solvent so as to obtain a clear solution in the 3-mL vial. The 3-mL vial with clear solution was then placed into a 20-mL vial containing 3 mL of anti-solvents. The 20-mL vial was sealed with a cap and kept at room temperature, allowing sufficient time for organic vapor to interact with the solution. After 2˜ 12 days, precipitates were isolated for XRPD analysis. The clear solution was transferred to 5° C., or further evaporated at room temperature. Solids were collected for XRPD analysis. The results summarized in Table 9 showed that crystals of Form A, Form B or Form C, as well as a gel-like material were obtained.

TABLE 9
Summary of liquid-vapor diffusion experiments

| Entry | Solvent | Anti-solvent | Solid form |
|---|---|---|---|
| Liq-Vap #1 | EtOH | Toluene | Form B* |
| Liq-Vap #2 | IPA | MTBE | Form A* |
| Liq-Vap #3 | MEK | n-Heptane | Form A |
| Liq-Vap #4 | MIBK | Toluene | gel |
| Liq-Vap #5 | IPAc | MTBE | Form A* |
| Liq-Vap #6 | EtOAc | n-Heptane | Form C |
| Liq-Vap #7 | DCM | MTBE | Form B |

*solids were obtained after 5° C. storage or further evaporation at RT.

### Example 5: Slurry Experiments at Room Temperature

Slurry conversion experiments were conducted at room temperature in different solvent systems. About 15 mg of crystals of Form A were suspended in 0.25˜0.3 mL of solvent in a HPLC vial. After the suspension was stirred magnetically at a speed of 750 rpm for 6 days at room temperature, the remaining solids were isolated for XRPD analysis. The results summarized in Table 10 showed that Crystals of Form A, Form B or Form D were generated.

TABLE 10
Summary of slurry experiments at room temperature

| Entry | Solvent (v/v) | Solid form |
|---|---|---|
| Slurry #1 | IPA | Form A |
| Slurry #2 | MTBE | Form A + Form D |
| Slurry #3 | Toluene | Form A |
| Slurry #4 | Anisole | Form B |
| Slurry #5 | IPAc | Form A |
| Slurry #6 | EtOAc/n-Heptane (1:1) | Form A |
| Slurry #7 | MIBK/n-Heptane (1:1) | Form A |
| Slurry #8 | EtOH/MTBE (1:4) | Form A |
| Slurry #9 | CHCl₃/MTBE (1:4) | Form A |
| Slurry #10 | 2-MeTHF/Toluene (1:4) | Form A |
| Slurry #11 | ACN/Toluene (1:4) | Form B |
| Slurry #12 | H₂O | Form A |
| Slurry #13 | EtOH/H₂O (970:30, | N/A |
| | a_{w} = 0.2) | |
| Slurry #14 | EtOH/H₂O (927:73, | N/A |
| | a_{w} = 0.4) | |
| Slurry #15 | EtOH/H₂O (855:145, | N/A |
| | a_{w} = 0.6) | |
| Slurry #16 | EtOH/H₂O (704:296, | Form A |
| | a_{w} = 0.8) | |
| Slurry #17 | EtOH | Form A |

N/A: ~30 mg of Form A (813908-05-A) was added into 0.25 mL of solvents (~120 mg/mL), and clear solution was still observed.

### Example 6: Slurry Experiments at 50° C.

Slurry conversion experiments were conducted at 50° C. in different solvent systems. About 20 mg of crystals of Form A were suspended in 0.25 of solvent in a HPLC vial. After the suspension was stirred magnetically at a speed of 750 rpm for 6 days at 50° C., the remaining solids were isolated for XRPD analysis. The results summarized in Table 11 showed that Crystals of Form A or Form B were generated.

TABLE 11
Summary of slurry experiments at 50° C.

| Entry | Solvent (v/v) | Solid form |
|---|---|---|
| Slurry 50° C. #1 | IPA | Form A |
| Slurry 50° C. #2 | MTBE | Form A |
| Slurry 50° C. #3 | Toluene | Form A |
| Slurry 50° C. #4 | Anisole | Form B |
| Slurry 50° C. #5 | IPAc | Form A |
| Slurry 50° C. #6 | EtOAc/n-Heptane (1:5) | Form A |
| Slurry 50° C. #7 | MIBK/n-Heptane (1:5) | Form A |
| Slurry 50° C. #8 | EtOH/MTBE (1:9) | Form A |
| Slurry 50° C. #9 | CHCl₃/MTBE (1:9) | Form A |
| Slurry 50° C. #10 | 2-MeTHF/Toluene (1:9) | Form A |
| Slurry 50° C. #11 | ACN/Toluene (1:9) | Form B |
| Slurry 50° C. #12 | H₂O | Form A |

### Example 7: Slow Evaporation Experiments

Slow evaporation experiments were performed under various conditions. For each experiment, about 15 mg of crystals of Form A were dissolved in 0.5˜1.5 mL of solvent in a 3-mL glass vial. If the solids were not dissolved completely, suspensions were filtered using a PTFE membrane (pore size of 0.45 μm), and the filtrates were used for the follow-up steps. The visually clear solutions were subjected to evaporation at room temperature with vials sealed by Parafilm® (3-5 pinholes). The solids were isolated for XRPD analysis, and the results summarized in Table 12 showed that crystals of Form A, Form B, Form C, Form E and a gel-like material were obtained.

TABLE 12
Summary of slow evaporation experiments

| Entry | Solvent | Solid form |
|---|---|---|
| Slow Evap #1 | MeOH | Form A |
| Slow Evap #2 | EtOH | Form A |
| Slow Evap #3 | IPA | Form A |
| Slow Evap #4 | Acetone | Form A |
| Slow Evap #5 | MEK | Form E |
| Slow Evap #6 | EtOAc | Form C |
| Slow Evap #7 | IPAc | Form C |
| Slow Evap #8 | THF | Form C |
| Slow Evap #9 | 2-MeTHF | gel |
| Slow Evap #10 | ACN | Form C |
| Slow Evap #11 | DCM | Form B |
| Slow Evap #12 | CHCl₃ | Form B |

### Example 8: Polymer-Induced Crystallization Experiments

Polymer-induced crystallization experiments were performed with two sets of polymer mixtures in 6 solvents, respectively. For each experiment, approximately 15 mg of crystals of Form A were dissolved in a solvent to obtain a clear solution in a 3-mL vial. About 2 mg of polymer mixture was added into the 3-mL glass vial. All the samples were subjected to evaporation at room temperature to induce crystallization. The solids were isolated for XRPD analysis. The results summarized in Table 13 showed that crystals of Form A, Form B, Form C and a gel-like material were generated.

TABLE 13
Summary of polymer-induced crystallization experiments

| Entry | Solvent | Polymer | Solid form |
|---|---|---|---|
| Polym #A1 | MeOH | Mixture A | Form A |
| Polym #A2 | EtOH | | Form A |
| Polym #A3 | Acetone | | Form C |
| Polym #A4 | DCM | | Form B |
| Polym #A5 | THF | | Form C |
| Polym #A6 | EtOAc | | Form C |
| Polym #B1 | IPA | Mixture B | Form A |
| Polym #B2 | EtOH | | Form A |
| Polym #B3 | MEK | | Form C |
| Polym #B4 | CHCl₃ | | Form B |
| Polym #B5 | 2-MeTHF | | gel |
| Polym #B6 | IPAc | | Form C |

Polymer mixture A: polyvinyl pyrrolidone (PVP), polyvinyl alcohol (PVA), polyvinylchloride (PVC), polyvinyl acetate (PVAC), hypromellose (HPMC), and methyl cellulose (MC) (mass ratio of 1:1:1:1:1:1)
Polymer mixture B: polycaprolactone (PCL), polyethylene glycol (PEG), poly(methyl methacrylate) (PMMA), sodium alginate (SA), and hydroxyethyl cellulose (HEC) (mass ratio of 1:1:1:1:1).

### Example 9: Slow Cooling Experiments

Slow cooling experiments were conducted in 10 solvent systems. For each experiment, approximately 20 mg of crystals of Form A were suspended in 0.5˜ 1 mL of solvent in a 3-mL glass vial at room temperature. The suspension was then heated to 50° C. with stirring, equilibrated for about two hours and filtered using a PTFE membrane (pore size of 0.45 μm). Filtrates were slowly cooled down to 5° C. at a rate of 0.1° C./min. The obtained solids were kept isothermally at 5° C. before being isolated for XRPD analysis. Clear solutions were transferred to evaporation at room temperature. The results summarized in Table 14 showed that crystals of Form A, Form C and gel-like material were generated.

TABLE 14
Summary of slow cooling experiments

| Entry | Solvent (v/v) | Solid form |
|---|---|---|
| Slow cooling #1 | IPA | Form A |
| Slow cooling #2 | MIBK | Form C* |
| Slow cooling #3 | EtOAc | Form C |
| Slow cooling #4 | IPAc | Form C* |
| Slow cooling #5 | Anisole | gel |
| Slow cooling #6 | Toluene | Form A |
| Slow cooling #7 | MTBE | Form A |
| Slow cooling #8 | EtOH/n-Heptane (1:4) | Form A |
| Slow cooling #9 | MEK/Toluene (1:4) | Form C* |
| Slow cooling #10 | CHCl₃/MTBE (1:4) | Form A |

*Gel was first observed after cooling, and then transformed to Form C after slow evaporation at RT.

### Example 10: Anti-Solvent Addition Experiments

A total of 20 anti-solvent addition experiments were carried out. Approximate 15 mg of starting material (813908-05-A) was dissolved in 0.3˜ 1.0 mL solvent to obtain a clear solution, and the solution was magnetically stirred followed by addition of 0.2 mL anti-solvent per step till precipitate appeared or the total volume of anti-solvent reached 15 mL. The obtained precipitate was isolated for XRPD analysis. Results in Table 15 showed that Types A/B/D, gel and amorphous/low crystallinity samples were obtained.

TABLE 15
Summary of anti-solvent addition experiments

| Entry | Solvent | Anti-solvent | Solid form |
|---|---|---|---|
| Anti-solvent #1 | IPA | MTBE | gel |
| Anti-solvent #2 | ACN | toluene | amorphous |
| Anti-solvent #3 | | H₂O | Form A |
| Anti-solvent #4 | EtOH | MTBE | Form A |
| Anti-solvent #5 | | n-Heptane | Form A |
| Anti-solvent #6 | | Toluene | amorphous |
| Anti-solvent #7 | | H₂O | Form A |
| Anti-solvent #8 | MIBK | MTBE | amorphous |
| Anti-solvent #9 | | n-Heptane | Form A |
| Anti-solvent #10 | | Toluene | low crystallinity* |
| Anti-solvent #11 | DCM | MTBE | Form B |
| Anti-solvent #12 | | n-Heptane | Form D |
| Anti-solvent #13 | | Toluene | low crystallinity* |
| Anti-solvent #14 | 2-MeTHF | MTBE | Form A |
| Anti-solvent #15 | | n-Heptane | Form A + Form D |
| Anti-solvent #16 | | Toluene | Form A |
| Anti-solvent #17 | EtOAc | MTBE | low crystallinity* |
| Anti-solvent #18 | | n-Heptane | Form A |
| Anti-solvent #19 | | Toluene | amorphous |
| Anti-solvent #20 | DMSO | H₂O | Form A |

*Several weak diffraction peaks were observed but the crystal form cannot be assigned.

### Example 11: Slurry Conversion of Form E and Form F to Form A

Excess amounts of crystals of Form A were added to 2 mL of IPA. The mixture was magnetically stirred at a speed of 750 rpm at room temperature overnight to obtain a saturated solution. The saturated solution was filtered on a PTFE membrane (0.45 μm) to remove the excess solids.

An equal-mass physical mixture of crystals of Form A and Form E (6 mg of each Form) was added into 0.5 mL of the pre-saturated IPA solution and magnetically stirred at a speed of 750 rpm for 48 h. Solids were isolated from the suspension by centrifugation and an XRPD test was performed (shown at FIG. 19(b)).

Similarly, an equal-mass physical mixture of crystals of Form A and Form F (6 mg of each Form) was added into 0.5 mL of the pre-saturated IPA solution and magnetically stirred at a speed of 750 rpm for 48 h. Solids were isolated from the suspension by centrifugation and an XRPD test was performed (shown at FIG. 19(c)).

In the case of both mixtures, only crystals of Form A were isolated after stirring for 48 h. Based on the slurry conversion results, Form A was considered to be the thermodynamically stable form at room temperature among the three anhydrate Forms (Form A, Form E and Form F).

### Example 12: Differential Scanning Calorimetry

Differential scanning calorimetry was conducted for each crystalline form using a TA Q200/Q2000 DSC from TA Instruments. For each analysis, the DSC cell/sample chamber was purged with ultra-high purity nitrogen gas. The sample crystal was placed into the bottom of a crimped aluminium pan, and measured against an empty reference pan. The heating rate was 10° C./min in a temperature range between room temperature and the desired temperature, as seen on each thermogram. The heat flow was plotted versus the measured sample temperature. The data were reported in units of watts/gram (“W/g”). The plots were made with the endothermic peaks pointing down. The DSC thermograms for Forms A to F were obtained and can be seen at FIGS. 7 to 12.

### Example 13: Thermogravimetric Analyses

TGA was conducted for each crystalline form using a TA Q500/Q5000 TGA from TA Instruments. For each analysis, the TGA cell/sample chamber was purged with ultra-high purity nitrogen gas. The sample crystal was placed into the bottom of an open aluminium pan. The heating rate was 10° C./min in a temperature range between room temperature and the desired temperature, as seen on each thermogram. The weight was plotted versus the measured sample temperature. The data were reported in % of the initial weight. The TGA thermograms for Forms A to F were obtained and can be seen at FIGS. 7 to 12.

### Example 14: Stability Tests for Form A

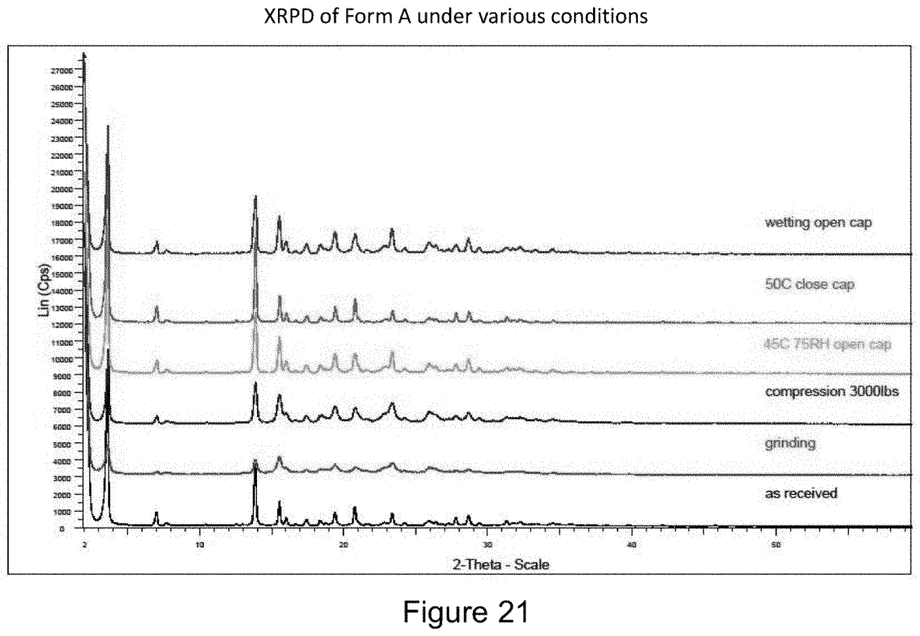

Experiments were conducted to evaluate the stability of Form A under various temperature, relative humidity and compression conditions. FIG. 21 shows XRPDs for Form A, recorded under various temperature, relative humidity and compression conditions.

It was shown that the crystalline structure remained generally similar on stressed samples compared to Form A as synthesized, suggesting stability of the crystal structure. Aggressive grinding for 2 minutes produced a minor decrease in the relative intensity of the peaks, possibly due to amorphization of a minor part of the sample.

### Example 15: Solidification from Melt

Experiments were conducted to evaluate potential crystal forms obtained by cooling a melt of Form A. Crystals of Form A were heated to 200° C. and then cooled to room temperature at a rate pf 10° C./min under nitrogen protection. The solid obtained after cooling was an amorphous form. The amorphous form thus obtained was then heated, and remained amorphous after heating to 110° C. Further heating to 130° C. then generated crystals of Form F. The DSC thermogram of the amorphous form showed two endotherms having peak temperatures at 125.7° C. and 154.3° C., and one exotherm having a peak temperature at 131.1° C.

X-Ray Diffractometers and Parameters

The XRPD measurements were performed using PANalytical X-ray powder diffractometers, that were used in reflection mode. The XRPD parameters that were used are listed in Table 16.

TABLE 16
Parameters for XRPD measurements

| | X′ Pert3 | X′ Pert3 | Empyrean |
|---|---|---|---|
| Parameters | (CPE-135) | (CPE-221) | (CPE-026) |
| Mode | Reflection | Reflection | VT-XRPD |
| | Cu, kα, | Cu, kα, | Cu, kα, |
| X-Ray wavelength | Kα1 (Å): 1.540598, | Kα1 (Å): 1.540598, | Kα1 (Å): 1.540598, |
| | Kα2 (Å): 1.544426 | Kα2 (Å): 1.544426 | Kα2 (Å): 1.544426 |
| | Kα2/Kα1 intensity | Kα2/Kα1 intensity | Kα2/Kα1 intensity |
| | ratio: 0.50 | ratio: 0.50 | ratio: 0.50 |
| X-Ray tube setting | 45 kV, 40 mA | 45 kV, 40 mA | 45 kV, 40 mA |
| Divergence slit | ⅛° | ⅛° | Automatic |
| Scan mode | Continuous | Continuous | Continuous |
| Scan range (°2TH) | 2.5°-40° | 2.5°-40° | 2°-40° |
| Scan step time (s) | 46.7 | 36.5 | 33.0 |
| Step size (°2TH) | 0.0263 | 0.0263 | 0.0167 |
| Test Time | About 5 min | About 4 min 30 s | About 10 min |

Although the invention has been illustrated and described with respect to one or more implementations, equivalent alterations and modifications will occur to others skilled in the art upon the reading and understanding of this specification. In addition, while a particular feature of the invention may have been disclosed with respect to only one of several implementations, such feature may be combined with one or more other features of the other implementations as may be desired and advantageous for any given or particular application.

Accordingly, it is understood that the examples and embodiments described herein are for illustrative purposes only and that various modifications or changes in light thereof will be suggested to persons skilled in the art and are to be included within the spirit and purview of this application and scope of the appended claims. Any publication, document, patent, patent application or publication referred to herein should be construed as incorporated by reference each in their entirety for all purposes.

1. A compound of Formula I: 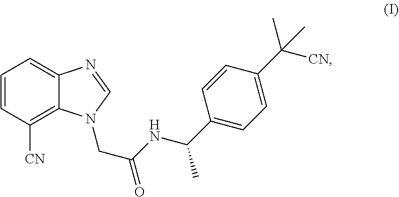 [SMILES: C[C@H](NC(=O)Cn1cnc2cccc(C#N)c21)c1ccc(C(C)(C)C#N)cc1]
which is crystalline and exhibits an X-ray powder diffraction (XRPD) pattern having characteristic peaks expressed in degrees 2Θ (±0.2° 2Θ) at 3.07, 5.96, 11.89, and 17.85.

2. The compound of claim 1, wherein the XRPD pattern further has characteristic peaks expressed in degrees 2Θ (±0.2° 2Θ) at 23.86 and 24.63.

3. The compound of claim 1 or 2, wherein the XRPD pattern further has characteristic peaks expressed in degrees 2Θ (±0.2° 2Θ) at 13.35, 14.90, 16.67, 20.08, 20.83, and 26.88.

4. The compound of any one of claims 1 to 3, wherein the XRPD pattern further has characteristic peaks expressed in degrees 2Θ (±0.2° 2Θ) at 8.92, 13.75, 22.15 and 39.20.

5. A compound of Formula I: 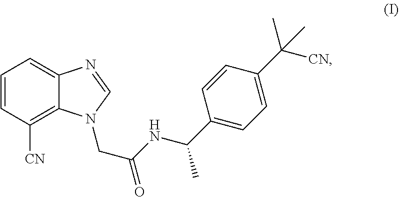 [SMILES: C[C@H](NC(=O)Cn1cnc2cccc(C#N)c21)c1ccc(C(C)(C)C#N)cc1]
which is crystalline and has a Differential Scanning Calorimetry (DSC) thermogram that exhibits an endotherm having a peak temperature of about 168.9° C.

6. A compound of Formula I: 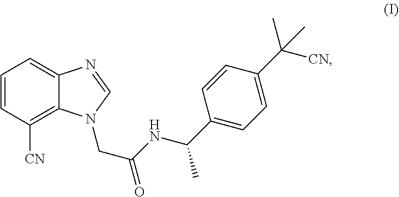 [SMILES: C[C@H](NC(=O)Cn1cnc2cccc(C#N)c21)c1ccc(C(C)(C)C#N)cc1]
having an X-ray powder diffraction pattern substantially the same as shown in FIG. 1.

7. The compound of any one of claims 1 to 6, that comprises one crystalline form at a purity of 95% or higher.

8. The compound of claim 7, wherein the purity is of 99% or higher.

9. The compound of claim 7, wherein the purity is of 99.8% or higher.

10. The compound of any one of claims 1 to 6, that is substantially pure.

11. Use of the compound of any one of claims 1 to 10, for the treatment of a nociceptive pain disorder.

12. Use of the compound of any one of claims 1 to 10, for the treatment of a chronic nociceptive pain disorder.

13. Use of the compound of any one of claims 1 to 10, for the treatment of osteoarthritis.

14. Use of the compound of any one of claims 1 to 10, for the treatment of tendinitis.

15. Use of the compound of any one of claims 1 to 10, for the treatment of chronic tendinitis.

16. Use of the compound of any one of claims 1 to 10, for the treatment of pelvic pain.

17. Use of the compound of any one of claims 1 to 10, for the treatment of neuropathic pain.

18. Use of the compound of any one of claims 1 to 10, for the treatment of peripheral neuropathy.

19. Use of the compound of any one of claims 1 to 10, for the treatment of postherpetic neuralgia (PHN).

20. Use of the compound of any one of claims 1 to 10, for the treatment of gastroesophageal reflux disease (GERD).

21. Use of the compound of any one of claims 1 to 10, for the treatment of diabetes.

22. Use of the compound of any one of claims 1 to 10, for the treatment of obesity.

23. Use of the compound of any one of claims 1 to 10, for the treatment of chronic cough.

24. Use of the compound of any one of claims 1 to 10, for the treatment of chronic obstructive pulmonary disease (COPD).

25. Use of the compound of any one of claims 1 to 10, for the treatment of irritable bowel syndrome (IBS).

26. Use of the compound of any one of claims 1 to 10, for the treatment of overactive bladder.

27. Use of the compound of any one of claims 1 to 10, for inhibiting vanilloid receptor 1 (VR1).

28. A method for the treatment of a nociceptive pain disorder, comprising administering the compound of any one of claims 1 to 10 to a subject in need thereof.

29. A method for the treatment of a chronic nociceptive pain disorder, comprising administering the compound of any one of claims 1 to 10 to a subject in need thereof.

30. A method for the treatment of osteoarthritis, comprising administering the compound of any one of claims 1 to 10 to a subject in need thereof.

31. A method for the treatment of tendinitis, comprising administering the compound of any one of claims 1 to 10 to a subject in need thereof.

32. A method for the treatment of chronic tendinitis, comprising administering the compound of any one of claims 1 to 10 to a subject in need thereof.

33. A method for the treatment of pelvic pain, comprising administering the compound of any one of claims 1 to 10 to a subject in need thereof.

34. A method for the treatment of neuropathic pain, comprising administering the compound of any one of claims 1 to 10 to a subject in need thereof.

35. A method for the treatment of peripheral neuropathy, comprising administering the compound of any one of claims 1 to 10 to a subject in need thereof.

36. A method for the treatment of postherpetic neuralgia (PHN), comprising administering the compound of any one of claims 1 to 10 to a subject in need thereof.

37. A method for the treatment of gastroesophageal reflux disease (GERD), comprising administering the compound of any one of claims 1 to 10 to a subject in need thereof.

38. A method for the treatment of diabetes, comprising administering the compound of any one of claims 1 to 10 to a subject in need thereof.

39. A method for the treatment of obesity, comprising administering the compound of any one of claims 1 to 10 to a subject in need thereof.

40. A method for the treatment of chronic cough, comprising administering the compound of any one of claims 1 to 10 to a subject in need thereof.

41. A method for the treatment of chronic obstructive pulmonary disease (COPD), comprising administering the compound of any one of claims 1 to 10 to a subject in need thereof.

42. A method for the treatment of irritable bowel syndrome (IBS), comprising administering the compound of any one of claims 1 to 10 to a subject in need thereof.

43. A method for the treatment of overactive bladder, comprising administering the compound of any one of claims 1 to 10 to a subject in need thereof.

44. A method for inhibiting vanilloid receptor 1 (VR1), comprising administering the compound of any one of claims 1 to 10 to a subject in need thereof.

45. A pharmaceutical composition, comprising a compound of any one of claims 1 to 10 and a pharmaceutically acceptable carrier or excipient.

46. The pharmaceutical composition of claim 45, that is formulated as an oral dosage form.

47. The pharmaceutical composition of claim 46, wherein the oral dosage form is a tablet, a capsule, a lozenge, a pastille or a granule.

48. The pharmaceutical composition of claim 45, that is formulated as an oral suspension.

49. Use of the pharmaceutical composition of any one of claims 45 to 48, for the treatment of a nociceptive pain disorder.

50. Use of the pharmaceutical composition of any one of claims 45 to 48, for the treatment of a chronic nociceptive pain disorder.

51. Use of the pharmaceutical composition of any one of claims 45 to 48, for the treatment of osteoarthritis.

52. Use of the pharmaceutical composition of any one of claims 45 to 48, for the treatment of tendinitis.

53. Use of the pharmaceutical composition of any one of claims 45 to 48, for the treatment of chronic tendinitis.

54. Use of the pharmaceutical composition of any one of claims 45 to 48, for the treatment of pelvic pain.

55. Use of the pharmaceutical composition of any one of claims 45 to 48, for the treatment of neuropathic pain.

56. Use of the pharmaceutical composition of any one of claims 45 to 48, for the treatment of peripheral neuropathy.

57. Use of the pharmaceutical composition of any one of claims 45 to 48, for the treatment of PHN.

58. Use of the pharmaceutical composition of any one of claims 45 to 48, for the treatment of GERD.

59. Use of the pharmaceutical composition of any one of claims 45 to 48, for the treatment of diabetes.

60. Use of the pharmaceutical composition of any one of claims 45 to 48, for the treatment of obesity.

61. Use of the pharmaceutical composition of any one of claims 45 to 48, for the treatment of chronic cough.

62. Use of the pharmaceutical composition of any one of claims 45 to 48, for the treatment of COPD.

63. Use of the pharmaceutical composition of any one of claims 45 to 48, for the treatment of IBS.

64. Use of the pharmaceutical composition of any one of claims 45 to 48, for the treatment of overactive bladder.

65. Use of the pharmaceutical composition of any one of claims 45 to 48, for inhibiting VR1.

66. A method for the treatment of a nociceptive pain disorder, comprising administering the pharmaceutical composition of any one of claims 45 to 48 to a subject in need thereof.

67. A method for the treatment of a chronic nociceptive pain disorder, comprising administering the pharmaceutical composition of any one of claims 45 to 48 to a subject in need thereof.

68. A method for the treatment of osteoarthritis, comprising administering the pharmaceutical composition of any one of claims 45 to 48 to a subject in need thereof.

69. A method for the treatment of tendinitis, comprising administering the pharmaceutical composition of any one of claims 45 to 48 to a subject in need thereof.

70. A method for the treatment of chronic tendinitis, comprising administering the pharmaceutical composition of any one of claims 45 to 48 to a subject in need thereof.

71. A method for the treatment of pelvic pain, comprising administering the pharmaceutical composition of any one of claims 45 to 48 to a subject in need thereof.

72. A method for the treatment of neuropathic pain, comprising administering the pharmaceutical composition of any one of claims 45 to 48 to a subject in need thereof.

73. A method for the treatment of peripheral neuropathy, comprising administering the pharmaceutical composition of any one of claims 45 to 48 to a subject in need thereof.

74. A method for the treatment of PHN, comprising administering the pharmaceutical composition of any one of claims 45 to 48 to a subject in need thereof.

75. A method for the treatment of GERD, comprising administering the pharmaceutical composition of any one of claims 45 to 48 to a subject in need thereof.

76. A method for the treatment of diabetes, comprising administering the pharmaceutical composition of any one of claims 45 to 48 to a subject in need thereof.

77. A method for the treatment of obesity, comprising administering the pharmaceutical composition of any one of claims 45 to 48 to a subject in need thereof.

78. A method for the treatment of chronic cough, comprising administering the pharmaceutical composition of any one of claims 45 to 48 to a subject in need thereof.

79. A method for the treatment of COPD, comprising administering the pharmaceutical composition of any one of claims 45 to 48 to a subject in need thereof.

80. A method for the treatment of IBS, comprising administering the pharmaceutical composition of any one of claims 45 to 48 to a subject in need thereof.

81. A method for the treatment of overactive bladder, comprising administering the pharmaceutical composition of any one of claims 45 to 48 to a subject in need thereof.

82. A method for inhibiting VR1, comprising administering the pharmaceutical composition of any one of claims 45 to 48 to a subject in need thereof.

83. A process for preparing the compound of any one of claims 1 to 6, comprising:
treating a salt of (S)-2-(7-Cyano-1H-benzimidazol-1-yl)-N-{1-[4-(1-cyano-1-methylethyl)phenyl]ethyl}acetamide with a base, to obtain a free base compound; and
crystallizing the free base compound to obtain the compound of Formula I.

84. The process of claim 83, wherein the salt is (S)-2-(7-Cyano-1H-benzimidazol-1-yl)-N-{1-[4-(1-cyano-1-methylethyl)phenyl]ethyl}acetamide hydrochloride.

85. The process of claim 83 or 84, wherein the base is selected from the group consisting of sodium bicarbonate, potassium bicarbonate, cesium bicarbonate, sodium carbonate, potassium carbonate and cesium carbonate.

86. The process of claim 85, wherein the base is sodium bicarbonate.

87. The process of any one of claims 83 to 86, wherein the base is solubilized in water.

88. The process of any one of claims 83 to 87, wherein the salt of (S)-2-(7-Cyano-1H-benzimidazol-1-yl)-N-{1-[4-(1-cyano-1-methylethyl)phenyl]ethyl}acetamide is solubilized in a solvent selected from the group consisting of methanol, ethanol, water and mixtures thereof.

89. The process of claim 88, wherein the solvent is a mixture of methanol and water.

90. The process of claim 88, wherein the solvent is methanol.

91. The process of any one of claims 83 to 90, wherein crystallizing the free base compound comprises:
solubilizing the free base compound by heating to obtain a free base solution;
cooling the free base solution to room temperature to obtain a free base slurry; and
filtering the free base slurry to obtain the compound of Formula I.

92. The process of claim 91, wherein the free base is solubilized in a mixture of water and methanol.

93. The process of claim 91 or 92, wherein filtering the free base slurry comprises:
obtaining a filter cake of the free base; and
drying the filter cake to obtain the compound of Formula I.

94. The process of claim 93, wherein drying the filter cake comprises drying the filter cake under vacuum at a temperature between about 40° C. and 45° C.
